(19) United States
(12) Patent Application Publication
(10) Pub. No.: US 2006/0019937 A1
(43) Pub. Date: Jan. 26, 2006

(54) Novel crystalline forms of 6alpha, 9alpha -difluoro-11beta-hydroxy-16alpha-methyl-3-oxo-17alpha-propionyloxy-androsta-1,4-diene 17beta-carboxylic acid and processes for preparation thereof

(76) Inventors: Itai Adin, Beer-Sheva (IL); Carmen Iustain, Beer-Sheva (IL); Yuri Futerman, Beer Sheva (IL)

Correspondence Address: Martin D. Moynihan;PRTSI, Inc., P.O. Box 16446, Arlington, VA, 22215, US

(21) Appl. No.: 11/188,839

(22) Filed: Jul. 26, 2005

Related U.S. Application Data
(60) Provisional application No. 60/590,920, filed on Jul. 26, 2004.
(60) Provisional application No. 60/599,875, filed on Aug. 10, 2004.

(51) Int. Cl. A61K 31/573

(52) U.S. Cl. 514/179

(57) ABSTRACT

Novel crystalline forms II, III, IV, V, VI, VII and VIII of 6α,9α-difluoro-11β-hydroxy-16α-methyl-3-oxo-17α-propionyloxyandrosta-1,4-diene-17β-carboxylic acid, a chemical intermediate useful in the preparation of fluticasone propionate, and novel methods of making these forms, substantially free of water, are disclosed.

### RELATED APPLICATIONS

The present application claims the benefit of U.S. Provisional Patent Application No. 60/590,920, filed on Jul. 26, 2004, and U.S. Provisional Patent Application No. 60/599,875, filed on Aug. 10, 2004, which are incorporated herein by reference.

### FIELD OF THE INVENTION

The present invention relates to novel, crystalline forms of 6α,9α-difluoro-11β-hydroxy-16α-methyl-3-oxo-17α-propionyloxyandrosta-1,4-diene-17β-carboxylic acid, a chemical intermediate useful in the preparation of fluticasone propionate, and of solvates thereof, and methods for producing same.

### BACKGROUND OF THE INVENTION

6α,9α-difluoro-11β-hydroxy-16α-methyl-3-oxo-17α-propionyloxyandrosta-1,4-diene-17β-carboxylic acid (Compound I): 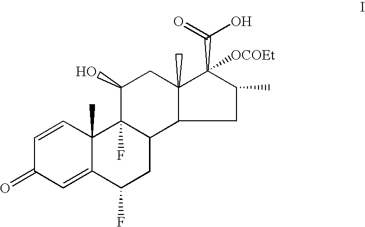
is a chemical compound known to be useful in the preparation of S-fluoromethyl-6α,9α-difluoro-11β-hydroxy-16α-methyl-3-oxo-17α-propionyloxy androsta-1,4-diene-17β-carbothioate (fluticasone propionate, Compound II):

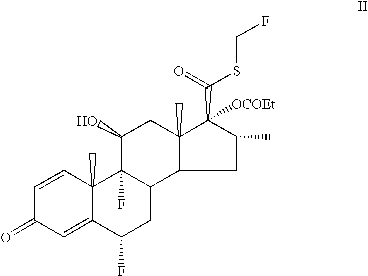

S-fluoromethyl-6α,9α-difluoro-11β-hydroxy-16α-methyl-3-oxo-17α-propionyloxy androsta-1,4-diene-17β-carbothioate, also known as fluticasone propionate, is a steroidal anti-inflammatory agent, particularly useful for the treatment of respiratory disorders, like asthma. Presently, fluticasone propionate is available commercially in the USA under the brand names Flovent diskus™, Advair discus™, Flonase™ and Cutivate™.

Various synthetic routes for preparing fluticasone propionate and the intermediates thereof were previously described in U.S. Pat. Nos. 4,335,121 and 6,747,163; in U.S. patent application Ser. Nos. 09/513,399 and 10/406,310 published as U.S. 2002/0133032 and 2004/0116396; PCT Patent Applications EP03/01116 and IN03/000219 published as WO2003/066654 and WO2004/001369 respectively; British patent GB 2,088,877; and Israeli patent IL 109,656, which are incorporated by reference as if fully set forth herein.

A process for preparing fluticasone propionate, as described in U.S. Pat. No. 4,335,121, is depicted in Scheme 1:

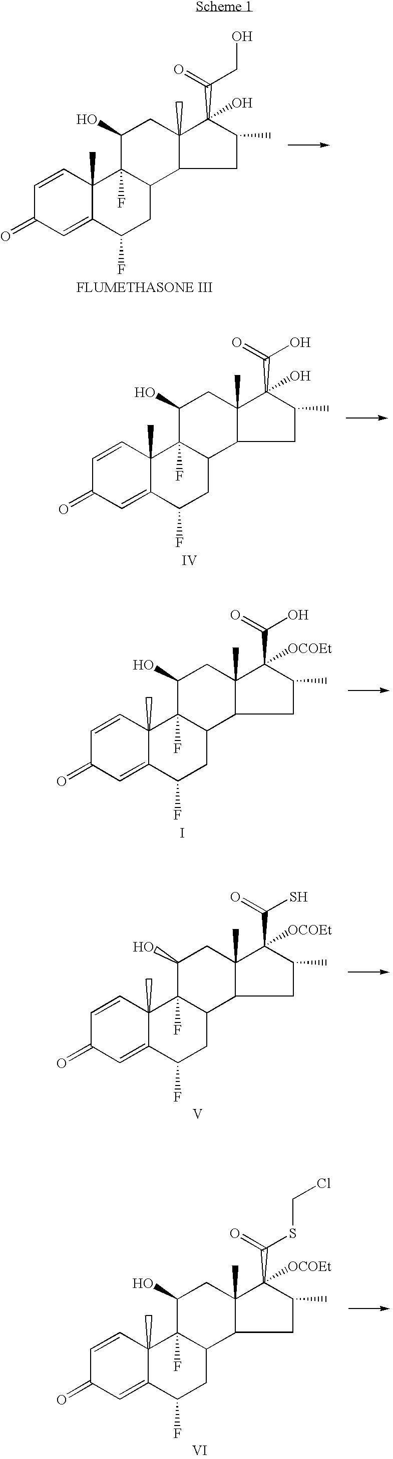

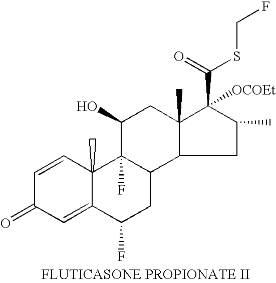

The process comprising converting 6α,9α-difluoro-11β,17α-dihydroxy-16α-methyl-3-oxoandrosta-1,4-diene-17β-carboxylic acid (Compound IV) to Compound I by reaction with propionyl chloride or propionyl anhydride, in the presence of non-hydroxylic solvent, e.g., chloroform, dichloromethane and benzene, followed by precipitation of a crystalline product containing Compound I from an acidic aqueous solution and subsequently drying the obtained solid. Apart from reporting a melting point of between 220° C. and 225° C., U.S. Pat. No. 4,335,121 does not further characterize the Compound I containing crystalline product.

The inventors of the present invention have repeated the method of preparing Compound I in accordance with Scheme 1, as taught in U.S. Pat. No. 4,335,121, denoted hereinafter as Compound I form I, (see Example 1 in the Experimental Section), and have characterized the product with the help of powder X-ray diffraction (FIG. 1), infrared spectroscopy (FIG. 2), differential scanning calorimetry (DSC, FIG. 3), thermo gravimetric analysis (TGA) and Karl-Fischer titration. It has been found that the Compound I is a hydrate having water content higher than 2.5% by weight. Exhaustive drying of the Compound I hydrate at 60° C. under vacuum reduced the water content of the product to about 1% by weight, as determined by thermo gravimetric analysis (FIG. 4).

The inventors of the present invention have determined that the presence of water in the crystalline Compound I hydrate causes the Compound I to degrade relatively quickly, precluding long-term storage of the product. Furthermore, the high water content, which increases the degradation rate of Compound I, may potentially lower the yield in the subsequent synthetic steps.

In PCT Patent Application No. PCT/IN03/000219, published as WO 2004/001369, a synthesis of Compound I, similar to that discussed above is disclosed, including the conversion of Compound IV to Compound I by reaction with propionic anhydride in the presence of acetone, followed by precipitation of the product from acidic aqueous solution and subsequently drying the obtained solid until the water content reaches a level below 5% by weight. It is to be expected that such a product also degrades quickly.

In U.S. patent application Ser. No. 09/513,399, published as U.S. 2002/0133032, a synthesis of Compound I, similar to that discussed above is disclosed, including the conversion of Compound IV to Compound I by reaction with propionyl chloride in the presence of acetone, followed by precipitation of the product from acidic aqueous solution and subsequently drying the obtained solid for 12 hours at 60° C. The thus-dried product is re-crystallized from a 3-pentanone: 2-butanone: water solution (about 90:10:1). The thus-produced Compound I is not further characterized.

Solvates, in the context of the present invention, may be defined as aggregates that consist of one or more molecules of the compound with one or more solvent molecules. One common type of solvate is a hydrate (e.g., Compound I hydrate), in which a compound is aggregated with water. Solvates often have crystalline structure hence the solvent molecules as well as the compound molecules are ordered and make up part of the crystal lattice.

Many compounds and solvates are polymorphic, that is having the property of crystallizing in two or more forms with distinct structures. Each such structure is called a polymorph of the compound. Polymorphs have the same chemical composition but differ in packing and geometrical arrangement, and exhibit different physical properties such as melting point, shape, color, density, hardness, deformability, stability and dissolution. (see, for example, Theory and Origin of Polymorphism in “Polymorphism in Pharmaceutical Solids” (1999) ISBN: 8247-0237).

As is noted above Compound I, as described in the prior art, is a hydrate with a high water content, adversely affecting the utility and stability of the product. Efforts of removing the water by heating under vacuum require time and energy, rendering such methods economically unviable on an industrial scale.

Thus, there is a widely recognized need for, and it would be highly advantageous to have Compound I as a novel crystalline polymorph or solvate, devoid of the above limitations. Preferably such a Compound I would be useful in the preparation of fluticasone propionate.

### SUMMARY OF THE INVENTION

The present invention provides substantially anhydrous, novel crystalline forms and novel solvates of Compound I, which maintain their stability during storage, and which are suitable for use in the preparation of fluticasone propionate, and processes for the preparation thereof.

According to the teachings of the present invention there is provided a crystalline Compound I (including a solvate thereof), wherein the impurities content during the storage period thereof does not exceed 2%, preferably 1%, and more preferably 0.5% in respect to the total weight of the product.

According to the teachings of the present invention there is provided a crystalline Compound I (including a solvate thereof) containing water in an amount of less than about 2% w/w and preferably, less than about 1% w/w, and most preferably less than about 0.5% w/w, in respect to the total weight of the product.

As noted above, Compound I of the present invention is useful as an intermediate in preparing fluticasone propionate, having water content in an amount of less than about 2% w/w and preferably, less than about 1% w/w, and most preferably less than about 0.5% w/w, in respect to the total weight of the product.

Thus, according to the teachings of the present invention, there is provided a process for preparing fluticasone propionate, the process comprising: providing a crystalline Compound I (such as a solvate thereof) having a water content as defined above; and converting the crystalline Compound I to fluticasone propionate according to any method known in the art.

One embodiment of the present invention relates to novel crystalline forms of Compound I.

Another embodiment of the present invention relates to a novel preparation processes for preparing novel crystalline forms of Compound I.

Yet another embodiment of the present invention relates to improved methods of purifying Compound I.

Another embodiment of the present invention relates to a stable crystalline solid comprising Compound I form II, or form III, or form IV, or form V, or form VI, or form VII or form VIII or any mixture thereof.

Another embodiment of the present invention relates to a stable crystalline solid comprising Compound I form II, or form III, or form IV, or form V, or form VI, or form VII, or form VIII, or any mixture thereof, that have a water content of less than about 2% w/w and preferably, less than about 1% w/w, and most preferably less than about 0.5% w/w, in respect to the total weight of the product.

Yet another embodiment of the present invention relates to a stable crystalline solid comprising Compound I form II, which is characterized by unique powder X-ray diffraction pattern (Table 1, FIG. 5). The strong diffraction peaks at 7.3, 9.7, 13.6, 14.6 and 18.6±0.2 degrees 2 θ are most characteristic of this form.

Yet another embodiment of the present invention relates to a stable crystalline solid comprising Compound I form II, which is further characterized by a unique infra-red spectrum (FIG. 6). The absorption peaks at 3315, 3165, 2266 (C≡N group, originating from acetonitrile) and the pattern created by the peaks at 1732 and 1749±4 cm⁻¹ are most characteristic of this form.

Another embodiment of the present invention relates to a process for preparing stable crystalline solid comprising Compound I form II comprising the steps of: (a) dispersing Compound I in acetonitrile; and (b) isolating the Compound I form II.

Yet another embodiment of the present invention relates to stable crystalline solid comprising Compound I form II, wherein said Compound I form II is Compound I form II acetonitrile solvate.

Yet another embodiment of the present invention relates to a stable crystalline solid comprising Compound I form II, wherein said Compound I form II contains acetonitrile in amount of between about 5% to about 7% w/w.

Yet another embodiment of the present invention relates to a method of making stable crystalline solid comprising Compound I form VIII comprising the step of heating Compound I form II to about 150° C.

Yet another embodiment of the present invention relates to a stable crystalline solid comprising Compound I form III, which is characterized by unique powder X-ray diffraction pattern (Table 2, FIG. 9). The strong diffraction peaks at 13.9, 15.1, 23.6 and 28.4±0.2 degrees 2 θ are most characteristic of this form. Compound I form III is further characterized by having a melting point of 232-234° C.

Yet another embodiment of the present invention relates to a stable crystalline solid comprising Compound I form III, which is characterized by a unique infra-red spectrum (FIG. 10). The strong absorption peaks at 3547 and 1738 (resolved singlet) ±4 cm⁻¹ are most characteristic of this form.

Another embodiment of the present invention relates to a process for preparing stable crystalline solid comprising Compound I form III comprising the steps of: (a) dissolving Compound I in toluene or in 1-propanol to form a solution; (b) crystallizing Compound I Form III from the solution; and (c) isolating the Compound I Form III.

In another embodiment of the present invention, Compound I is added into stirred toluene or 1-propanol to form a suspension. The suspension is heated to form a solution. Preferably, the suspension is heated to about reflux temperature, thus making Compound I substantially soluble in the solvent.

Yet another embodiment of the present invention relates to a stable crystalline solid comprising Compound I form IV, which is characterized by unique powder X-ray diffraction pattern (Table 3, FIG. 13). The diffraction peaks at 6.7, 7.6, 7.8, 13.9, 15.8 and 17.2±0.2 degrees 2 θ are most characteristic of this form. Compound I form IV is further characterized by having a melting point of 221-224° C.

Yet another embodiment of the present invention relates to a stable crystalline solid comprising Compound I form IV, which is characterized by a unique infra-red spectrum (FIG. 14). The absorption peaks at 3477, 3412, 3271, the doublet at 1660-1670 and the pattern created by the peaks at 1700-1750±4 cm⁻¹ are most characteristic of this form.

Another embodiment of the present invention relates to a process for preparing stable crystalline solid comprising Compound I form IV comprising the steps of: (a) dissolving Compound I in isopropanol or in ethyl acetate to form a solution; (b) crystallizing Compound I form IV from the solution; and (c) isolating the Compound I Form IV.

In another embodiment of the present invention, Compound I is added into stirred isopropanol or ethyl acetate to form a suspension. The suspension is heated to form a solution. Preferably, the suspension is heated to about reflux temperature, thus making Compound I substantially soluble in isopropanol or in ethyl acetate.

Yet another embodiment of the present invention relates to a stable crystalline solid comprising Compound I form V, which is characterized by unique powder X-ray diffraction pattern (Table 4, FIG. 15). The diffraction peaks at 7.2, 9.6, 12.5, 13.6, 14.5 and 18.5±0.2 degrees 2 θ are most characteristic of this form.

Yet another embodiment of the present invention relates to a stable crystalline solid comprising Compound I form V, which is further characterized by a unique infra-red spectrum (FIG. 16). The peaks at 3286, 1749, 1728 and the singlet at 1612±4 cm⁻¹ are most characteristic of this form.

Another embodiment of the present invention relates to a process for preparing stable crystalline solid comprising Compound I form V comprising the steps of: (a) dissolving Compound I in acetone to form a solution; (b) crystallizing Compound I Form V from the solution; and (c) isolating the Compound I Form V.

In another embodiment of the present invention, Compound I is added into stirred acetone to form a suspension. The suspension is heated to form a solution. Preferably, the suspension is heated to about reflux temperature, thus making Compound I substantially soluble in acetone.

Yet another embodiment of the present invention relates to stable crystalline solid comprising Compound I form V, wherein said Compound I form V is Compound I form V acetone solvate.

Yet another embodiment of the present invention relates to stable crystalline solid comprising Compound I form V, wherein said Compound I form V contains acetone in an amount of between about 10% to about 12% w/w.

Yet another embodiment of the present invention relates to a method of making stable crystalline solid comprising Compound I form VIII comprising the step of heating Compound I form V to between about 110° C. to about 150° C.

Yet another embodiment of the present invention relates to a stable crystalline solid comprising Compound I form VI, which is characterized by unique powder X-ray diffraction pattern (Table 5, FIG. 19). The diffraction peaks at 6.6, 7.7, 12.6, 13.9, 15.1 and 18.8±0.2 degrees 20 are most characteristic of this form.

Yet another embodiment of the present invention relates to a stable crystalline solid comprising Compound I form VI, which is further characterized by a unique infra-red spectrum (FIG. 20). The absorption peaks at 3547, 3388, 3290 and 1738 (broad) ±4 cm⁻¹ are most characteristic of this form.

Another embodiment of the present invention relates to a process for preparing stable crystalline solid comprising Compound I form VI comprising the steps of: (a) dissolving Compound I in isopropanol to form a solution; (b) crystallizing Compound I Form VI from the solution by rapid removal of the solvent; and (c) isolating the Compound I Form VI.

In another embodiment of the present invention, Compound I is added into stirred isopropanol to form a suspension. The suspension is heated to form a solution. Preferably, the suspension is heated to about reflux temperature, thus making Compound I substantially soluble in isopropanol.

By rapid removing the solvent, in the context of this invention, it is meant removing the organic volatiles by one of the known in the art drying technologies including vacuum ovens, tray ovens, rotary ovens, rotary evaporators and fluidized bed dryers.

Another embodiment of the present invention relates to a second process for preparing Compound I form VI comprising the steps of: (a) dissolving Compound I in a mixture of isopropanol and a non-polar anti-solvent, preferably cyclohexane, to form a solution; (b) crystallizing Compound I Form VI from the solution; and (c) isolating the Compound I Form VI.

In another embodiment of the present invention, Compound I is added into a stirred mixture of isopropanol and a non-polar anti-solvent to form a suspension. The suspension is heated to form a solution. Preferably, the suspension is heated to about reflux temperature, thus making Compound I substantially soluble in the mixture of isopropanol and the non-polar anti-solvent.

Yet another embodiment of the present invention relates to stable crystalline solid comprising Compound I form VI, wherein said Compound I form VI is Compound I form VI isopropanol solvate.

Yet another embodiment of the present invention relates to stable crystalline solid comprising Compound I form VI, wherein said Compound I form VI contains isopropanol in an amount of between about 6% to about 8% w/w.

Yet another embodiment of the present invention relates to a method of making Compound I form VIII comprising the step of heating Compound I form VI to between about 150° C. to about 160° C.

Yet another embodiment of the present invention relates to a stable crystalline solid comprising Compound I form VII, which is characterized by unique powder X-ray diffraction pattern (Table 6, FIG. 23). The diffraction peaks at 10.6, 11.0, 12.4, 14.9, 22.3 and 23.0±0.2 degrees 2 θ are most characteristic of this form.

Yet another embodiment of the present invention relates to a stable crystalline solid comprising Compound I form VII, which is further characterized by a unique infra-red spectrum (FIG. 24). The absorption peaks at 3468, 1740, 1703, 1063 and 1032±4 cm⁻¹ are most characteristic of this form.

Another embodiment of the present invention relates to a process for preparing stable crystalline solid comprising Compound I form VII comprising the steps of: (a) dissolving Compound I in ethyl acetate to form a solution; (b) crystallizing Compound I Form VII from the solution by rapid removal of the solvent; and (c) isolating the Compound I Form VII.

In another embodiment of the present invention, Compound I is added into stirred ethyl acetate to form a suspension. The suspension is heated to form a solution. Preferably, the suspension is heated to about reflux temperature, thus making Compound I substantially soluble in ethyl acetate.

Yet another embodiment of the present invention relates to stable crystalline solid comprising Compound I form VII, wherein said Compound I form VII is Compound I form VII ethyl acetate solvate.

Yet another embodiment of the present invention relates to stable crystalline solid comprising Compound I form VII, wherein said Compound I form VII contains ethyl acetate in an amount of between about 8% to about 14% w/w.

Yet another aspect the present invention relates to a method of making stable crystalline solid comprising Compound I form VIII comprising the step of heating Compound I form VII to about 100° C. to about 160° C.

Yet another embodiment of the present invention relates to a stable crystalline solid comprising Compound I form VIII, which is characterized by unique powder X-ray diffraction pattern (Table 7, FIG. 27). The diffraction peaks at 7.4, 10.0, 13.2, 13.9 and 15.4±0.2 degrees 2 θ are most characteristic of this form. Compound I form VIII is further characterized by having a melting point of 223-225° C.

Yet another embodiment of the present invention relates to a stable crystalline solid comprising Compound I form VIII, which is further characterized by a unique infra-red spectrum (FIG. 28). The absorption peaks at 3288, 1743, 1702, 1664 (singlet) and the doublet around 895±4 cm⁻¹ are most characteristic of this form.

Another embodiment of the present invention relates to a process for preparing stable crystalline solid comprising Compound I form VIII comprising the step of heating Compound I form II, or form V, or form VI or form VII.

Another embodiment of the present invention relates to stable crystalline solid comprising Compound I form II, or form III, or form IV, or form V, or form VI, or form VII, or form VIII, or any mixture thereof, that are stable during storage.

By storage-stable, in the context of the present invention, is meant Compound I crystalline form that have improved shelf-life without exhibiting a significant change in the active component's physico-chemical characteristics.

As used herein, storage-stable refers to retention of the original physico-chemical characteristics of Compound I crystalline form of the present invention over a period of at least one month, preferably over a period of 6 months and more preferably over periods exceeding one year. The impurity content in Compound I crystalline form over the storage period does not exceed 2%, preferably 1%, and more preferably 0.5% in respect to the total weight of the product.

In a stability test that was carried out to a sample of Compound I form II it has been found that the purity of the material (as determined by HPLC) was not changed during a storage period of 1 month, 6 months and even a storage period exceeding one year in a closed vessel at humidity of warehouse conditions and at room temperature.

Another embodiment of the present invention relates to a method of purifying Compound I by crystallization.

The term impure Compound I, as used herein, refers to a Compound I isolated by any process conventionally known in the art or to be developed in the future.

### BRIEF DESCRIPTION OF THE FIGURES

FIG. 1 shows powder X-ray diffraction of “Compound I” form I;

FIG. 2 shows infra-red spectrum of “Compound I” form I;

FIG. 3 shows DSC curve of “Compound I” form I;

FIG. 4 shows TGA curve of “Compound I” form I;

FIG. 5 shows powder X-ray diffraction of “Compound I” form II;

FIG. 6 shows infra-red spectrum of “Compound I” form II;

FIG. 7 shows DSC curve of “Compound I” form II;

FIG. 8 shows TGA curve of “Compound I” form II;

FIG. 9 shows powder X-ray diffraction of “Compound I” form III;

FIG. 10 shows infra-red spectrum of “Compound I” form III;

FIG. 11 shows DSC curve of “Compound I” form III;

FIG. 12 shows TGA curve of “Compound I” form III;

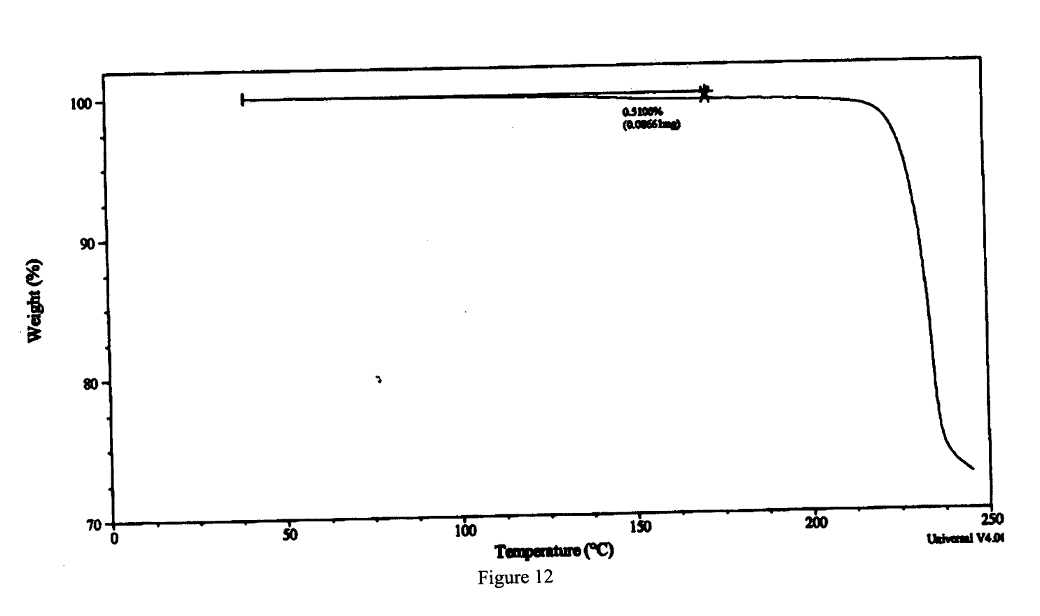

FIG. 13 shows powder X-ray diffraction of “Compound I” form IV;

FIG. 14 shows infra-red spectrum of “Compound I” form IV;

FIG. 15 shows powder X-ray diffraction of “Compound I” form V;

FIG. 16 shows infra-red spectrum of “Compound I” form V;

FIG. 17 shows DSC curve of “Compound I” form V;

FIG. 18 shows TGA curve of “Compound I” form V;

FIG. 19 shows powder X-ray diffraction of “Compound I” form VI;

FIG. 20 shows infra-red spectrum of “Compound I” form VI;

FIG. 21 shows DSC curve of “Compound I” form VI;

FIG. 22 shows TGA curve of “Compound I” form VI;

FIG. 23 shows powder X-ray diffraction of “Compound I” form VII;

FIG. 24 shows infra-red spectrum of “Compound I” form VII;

FIG. 25 shows DSC curve of “Compound I” form VII;

FIG. 26 shows TGA curve of “Compound I” form VII;

FIG. 27 shows powder X-ray diffraction of “Compound I” form VIII;

FIG. 28 shows infra-red spectrum of “Compound I” form VIII;

FIG. 29 shows DSC curve of “Compound I” form VIII; and

FIG. 30 shows TGA curve of “Compound I” form VIII.

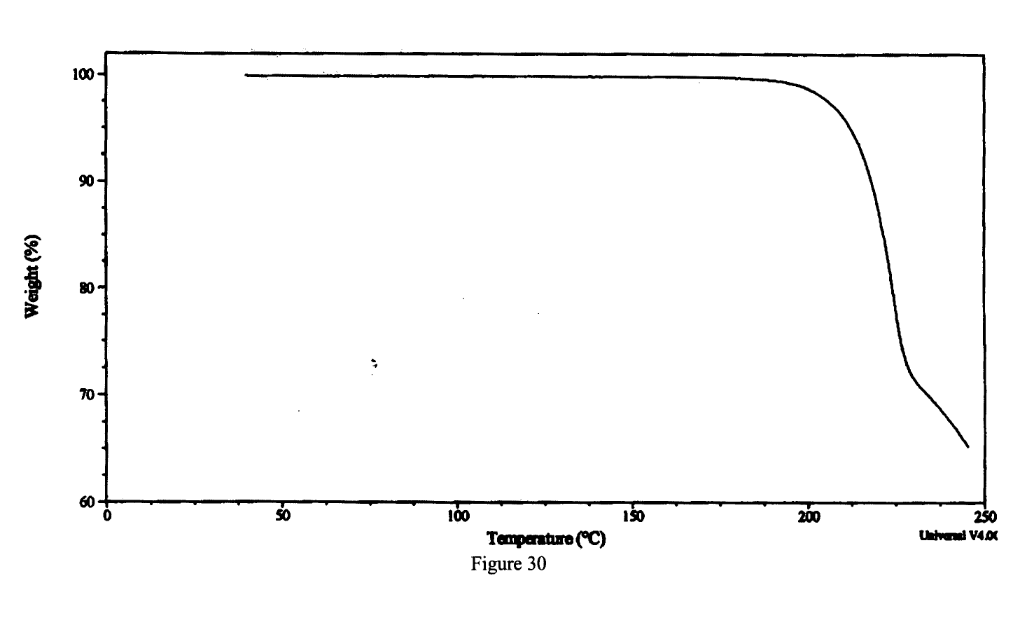

### DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The present invention provides novel polymorphs of 6α,9α-Difluoro-11β-hydroxy-16α-methyl-3-oxo-17α-propionyloxy androsta-1,4-diene-17β-carboxylic acid (“Compound I”) and new preparation processes thereof.

The present invention provides also novel polymorphs of Compound I, which have improved shelf life without exhibiting a significant change in the active component's physico-chemical characteristics.

The present invention provides also novel methods of purifying Compound I, preferably by crystallization.

Compound I used as the starting material in the embodiments disclosed hereinafter, is known and obtainable e.g. by conventional methods known in the art.

In an embodiment of the present invention a stable crystalline solid comprising Compound I, designated as Compound I form II is provided.

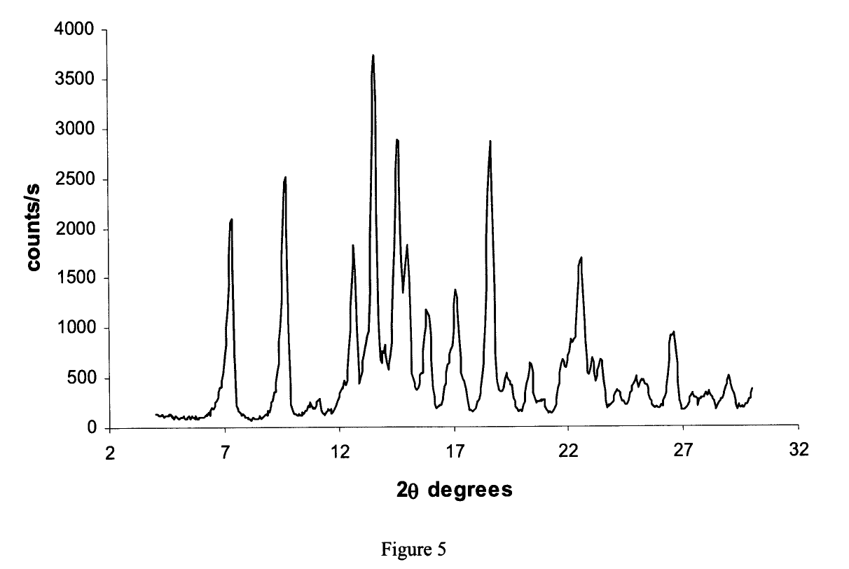

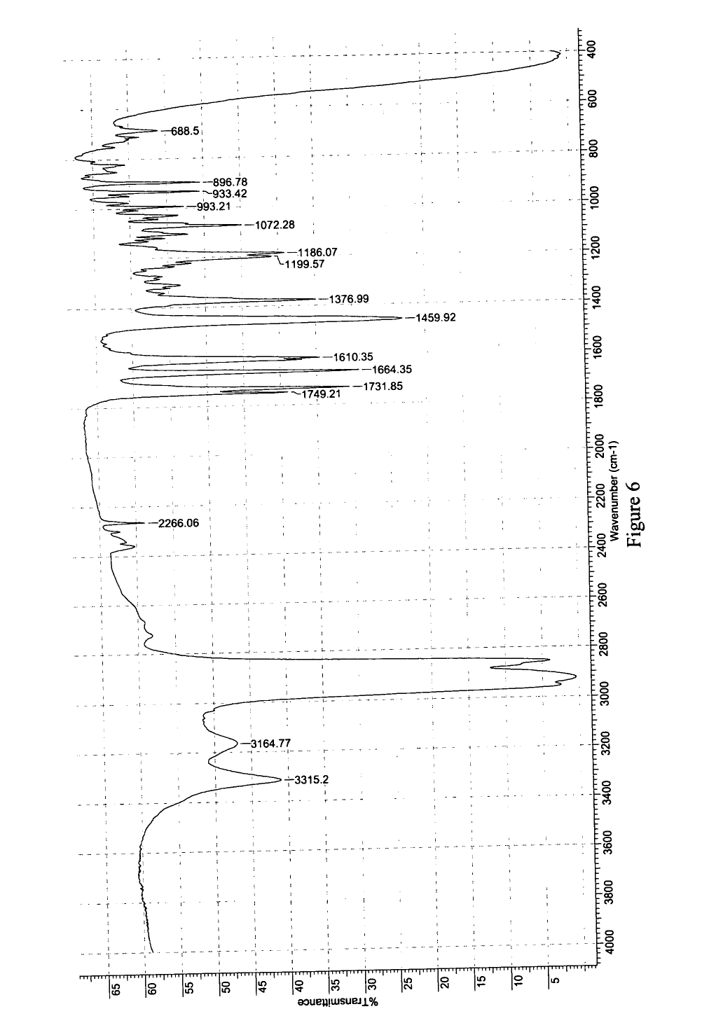

The stable crystalline solid comprising Compound I form II is characterized by unique powder X-ray diffraction pattern (Table 1, FIG. 5). The strong diffraction peaks at 7.3, 9.7, 13.6, 14.6 and 18.6±0.2 degrees 2 θ are most characteristic of this form. It is further characterized by a unique infra-red spectrum (FIG. 6). The absorption peaks at 3315, 3165, 2266 (C≡N group, originating from acetonitrile) and the pattern created by the peaks at 1732 and 1749±4 cm⁻¹ are most characteristic of this form.

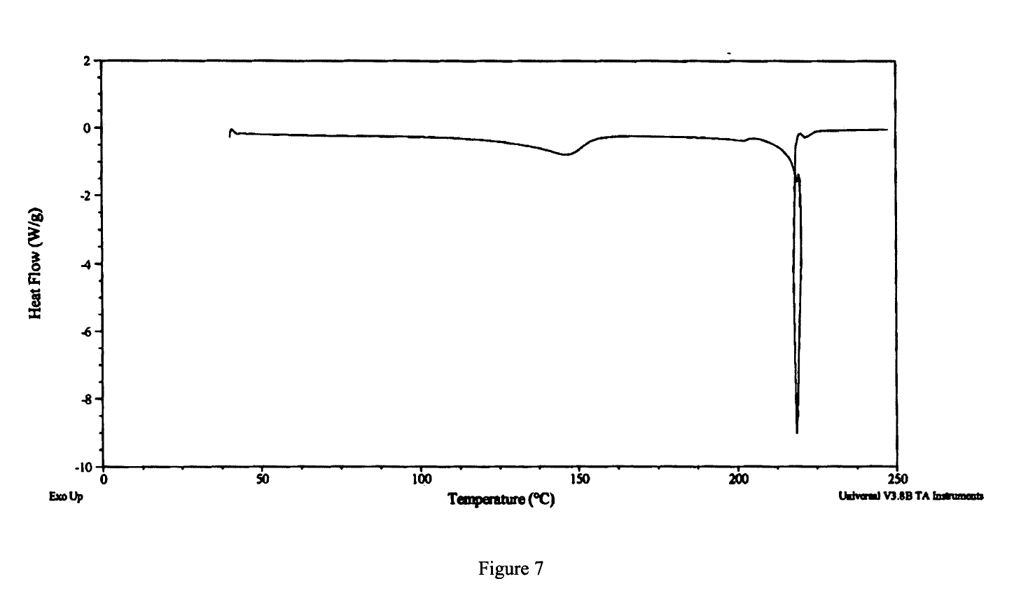

The stable crystalline solid comprising Compound I form II is further characterized by Differential Scanning Calorimetry (DSC). DSC curve of Compound I form II (FIG. 7) shows an endothermic peak around 150° C.

In another embodiment of the present invention a process for preparing Compound I form II is provided. The process comprising the steps of: (a) dispersing Compound I in acetonitrile; and (b) isolating the Compound I form II.

In another embodiment of the present invention, the dispersion is kept at a temperature in the range of from about 10° C. to about 50° C., more preferably from about 15° C. to about 30° C., most preferably at an ambient temperature.

In another embodiment of the present invention, the dispersion is stirred. The dispersion may be stirred by methods well known in the art, such as magnetic stirrer and mechanical stirrer.

In another embodiment of the present invention, the dispersion is stirred from about several hours to about several days, preferably the dispersion is stirred overnight.

In another embodiment of the present invention, the stable crystalline solid comprising Compound I form II is separated by techniques well-known in the art. Non-limiting examples of separation techniques, usable in context of the present invention, include filtering, vacuum filtration, decanting and centrifugation, filtering being the most preferred method.

In yet another embodiment of the present invention, the isolated stable crystalline solid comprising Compound I form II can be dried using conventionally known methods.

The drying stage may be carried out by increasing the temperature or reducing the pressure or a combination of both. Non limiting examples of drying technologies or equipments, usable in context of the present invention, include vacuum ovens, tray ovens, rotary ovens, rotary evaporators and fluidized bed dryers.

In yet another embodiment of the present invention, the stable crystalline solid comprising Compound I form II, obtained by the process disclosed hereinabove, is a solvate of acetonitrile.

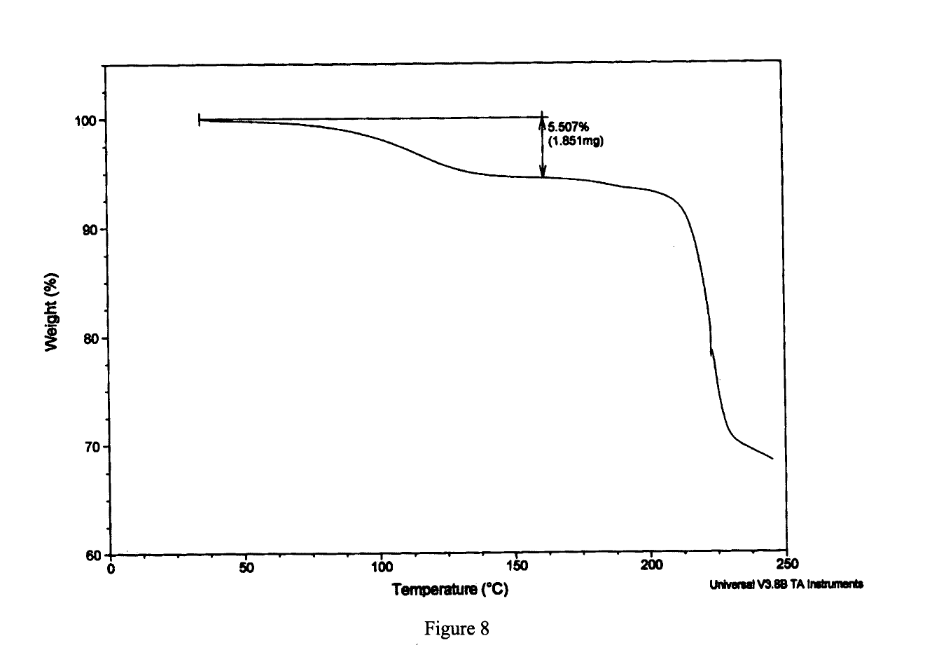

In yet another embodiment of the present invention the stable crystalline solid comprising Compound I form II, obtained by the process disclosed hereinabove, contains acetonitrile in an amount of between about 5% to about 7% w/w. Weight loss of from about 7% w/w is observed in thermo gravimetric analysis (TGA) (FIG. 8), and the identity of the released solvent was independently determined using head-space GC analysis.

TABLE 1
Form II - Powder X-ray diffraction peak positions and intensities

| Rel. | Peak | Rel. | Peak |
|---|---|---|---|
| Intensity | Position | Intensity | Position |
| (%) | (2θ deg) | (%) | (2θ deg) |
| 57 | 7.3 | 8 | 20.8 |
| 69 | 9.7 | 18 | 21.8 |
| 7 | 10.7 | 46 | 22.6 |
| 8 | 11.1 | 19 | 23.0 |
| 49 | 12.7 | 18 | 23.4 |
| 100 | 13.6 | 10 | 24.1 |
| 22 | 14.0 | 14 | 25.0 |
| 78 | 14.6 | 13 | 25.2 |
| 49 | 15.0 | 25 | 26.6 |
| 32 | 15.9 | 9 | 27.4 |
| 37 | 17.1 | 10 | 28.1 |
| 77 | 18.7 | 14 | 29.0 |
| 14 | 19.3 | 16 | 30.2 |
| 17 | 20.4 | | |

In another embodiment of the present invention a stable crystalline solid comprising Compound I, designated as Compound I form III is provided.

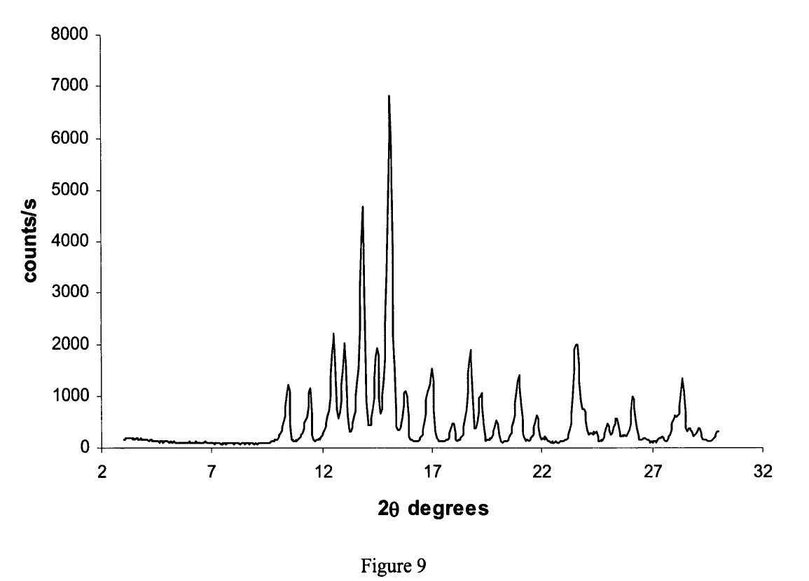

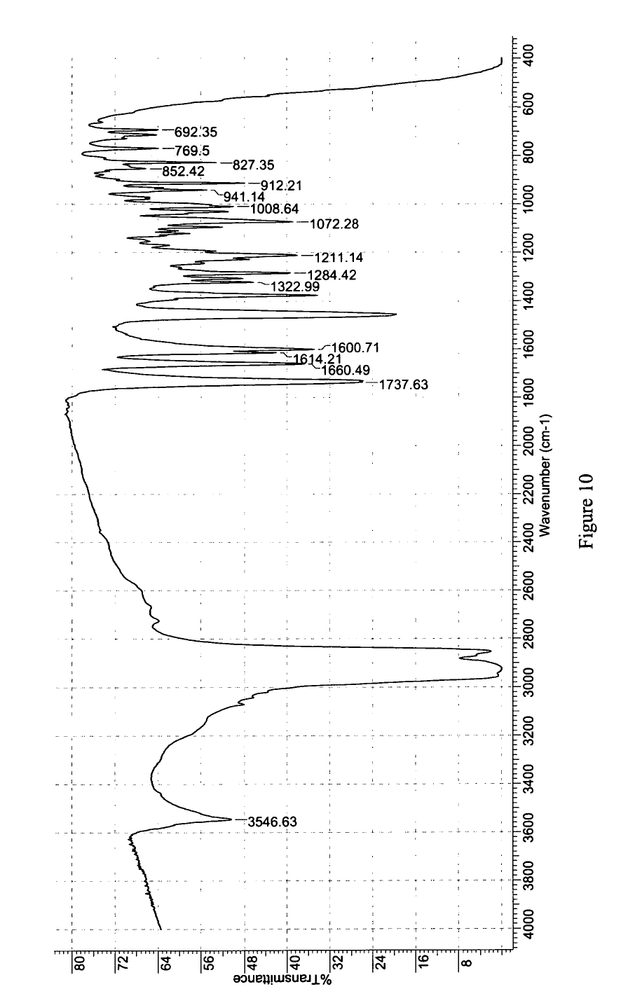

The stable crystalline solid comprising Compound I form III is characterized by unique powder X-ray diffraction pattern (Table 2, FIG. 9). The strong diffraction peaks at 13.9, 15.1, 23.6 and 28.4±0.2 degrees 2 θ are most characteristic of this form. The stable crystalline solid comprising Compound I form III is further characterized by having a melting point of 232-234° C. and unique infra-red spectrum (FIG. 10). The strong absorption peaks at 3547 and 1738 (resolved singlet) ±4 cm⁻¹ are most characteristic of this form.

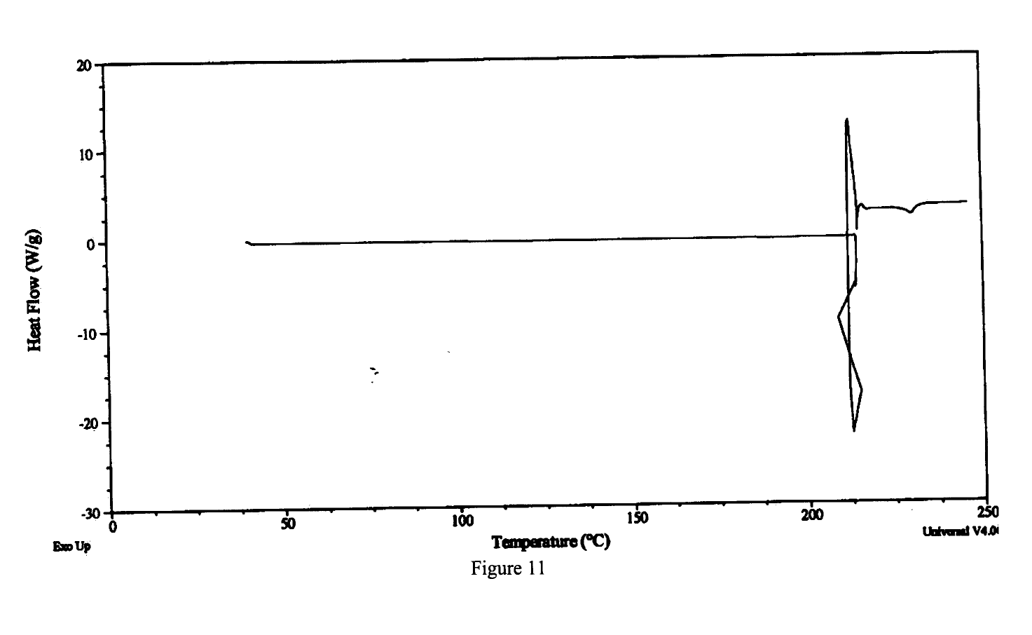

DSC curve of the stable crystalline solid comprising Compound I form III (FIG. 11) shows only an endothermic peak around 230° C. corresponding to its melting and decomposition.

In another embodiment of the present invention, a process for preparing stable crystalline solid comprising Compound I form III is provided. The process comprising the steps of: (a) dissolving Compound I in a solvent selected from a group consisting of toluene and 1-propanol to form a solution; (b) crystallizing Compound I Form III from the Compound I solution; and (c) isolating the Compound I Form III.

In another embodiment of the present invention, Compound I is added into stirred toluene or 1-propanol to form a suspension. The suspension is heated to form a solution. Preferably, the suspension is heated to about reflux temperature, thus making Compound I substantially soluble in the solvent.

In yet another embodiment of the present invention the solvent is toluene or 1-propanol.

In yet another embodiment of the present invention, the solution is kept under reflux from about several minutes to about several hours. Preferably the solution is kept under reflux for several minutes.

In another embodiment of the present invention, the solution is cooled to about room temperature.

In yet another embodiment of the present invention, the cooled solution is kept at room temperature until precipitation occurs.

In yet another embodiment of the present invention, Compound I form III is separated by techniques well-known in the art.

In yet another embodiment of the present invention, the isolated Compound I form III can be dried using conventionally known methods, preferably, the isolated Compound I form III is dried by placing it in a hood.

TABLE 2
Form III - Powder X-ray diffraction peak positions and intensities

| Rel. | Peak | Rel. | Peak |
|---|---|---|---|
| Intensity | Position | Intensity | Position |
| (%) | (2θ deg) | (%) | (2θ deg) |
| 18 | 10.5 | 30 | 23.6 |
| 17 | 11.4 | 5 | 24.4 |
| 33 | 12.5 | 7 | 25.0 |
| 30 | 13.0 | 8 | 25.4 |
| 69 | 13.9 | 15 | 26.1 |
| 28 | 14.5 | 3 | 26.7 |
| 100 | 15.1 | 3 | 27.4 |
| 16 | 15.8 | 9 | 28.0 |
| 22 | 17.0 | 20 | 28.4 |
| 7 | 17.9 | 6 | 28.7 |
| 28 | 18.8 | 6 | 29.1 |
| 16 | 19.2 | 5 | 29.9 |
| 8 | 19.9 | 4 | 30.3 |
| 20 | 20.9 | | |
| 9 | 21.8 | | |
| 3 | 22.1 | | |

In another embodiment of the present invention a stable crystalline solid comprising Compound I, designated as Compound I form IV is provided.

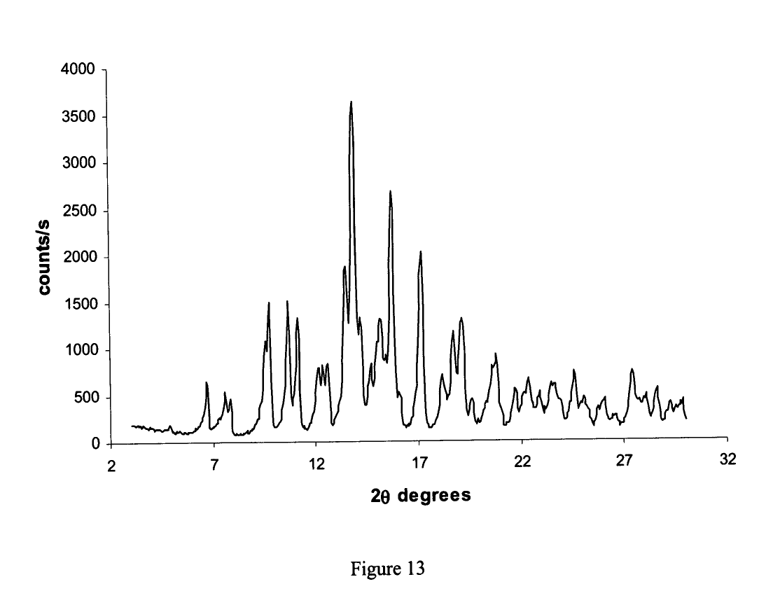

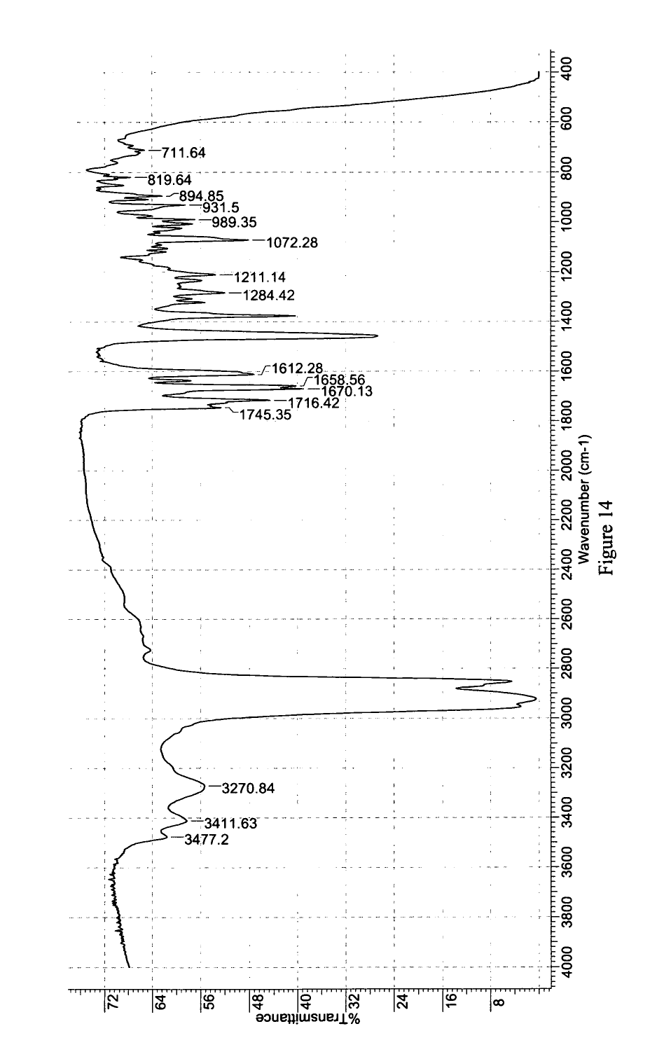

The stable crystalline solid comprising Compound I form IV is characterized by unique powder X-ray diffraction pattern (Table 3, FIG. 13). The diffraction peaks at 6.7, 7.6, 7.8, 13.9, 15.8 and 17.2±0.2 degrees 2 θ are most characteristic of this form. The stable crystalline solid comprising Compound I form IV is further characterized by having a melting point of 221-224° C. and unique infra-red spectrum (FIG. 14). The absorption peaks at 3477, 3412, 3271, the doublet at 1660-1670 and the pattern created by the peaks at 1700-1750±4 cm⁻¹ are most characteristic of this form.

DSC curve of the stable crystalline solid comprising Compound I Form IV (data not shown) shows only an endothermic peak around 220° C., corresponding to its melting and decomposition.

In another embodiment of the present invention, a process for preparing a stable crystalline solid comprising Compound I form IV is provided. The process comprising the steps of: (a) dissolving Compound I in isopropanol or in ethyl acetate to form a solution; (b) crystallizing Compound I Form IV from the solution; and (c) isolating the Compound I Form IV.

In another embodiment of the present invention, Compound I is added into stirred isopropanol or ethyl acetate to form a suspension. The suspension is heated to form a solution. Preferably, the suspension is heated to about reflux temperature, thus making Compound I substantially soluble in isopropanol or in ethyl acetate.

In yet another embodiment of the present invention, the solution is kept under reflux from about several minutes to about several hours. Preferably the solution is kept under reflux for several minutes.

In another embodiment of the present invention, the solution is cooled to about room temperature.

In yet another embodiment of the present invention, the cooled solution is kept at room temperature until precipitation occurs.

In yet another embodiment of the present invention, Compound I form IV is separated by techniques well-known in the art.

In yet another embodiment of the present invention, the isolated Compound I form IV may be dried using conventionally known methods. Preferably, the isolated Compound I form IV may be dried by placing it in a hood.

TABLE 3
Form IV - Powder X-ray diffraction peak positions and intensities

| Rel. | Peak | Rel. | Peak |
|---|---|---|---|
| Intensity | Position | Intensity | Position |
| (%) | (2θ deg) | (%) | (2θ deg) |
| 5 | 4.9 | 33 | 18.7 |
| 18 | 6.7 | 36 | 19.1 |
| 15 | 7.6 | 12 | 19.6 |
| 13 | 7.8 | 25 | 20.8 |
| 41 | 9.8 | 15 | 21.7 |
| 42 | 10.7 | 18 | 22.4 |
| 36 | 11.2 | 14 | 22.9 |
| 22 | 12.1 | 17 | 23.5 |
| 23 | 12.6 | 21 | 24.6 |
| 52 | 13.5 | 13 | 25.1 |
| 100 | 13.9 | 12 | 26.0 |
| 23 | 14.7 | 7 | 26.6 |
| 36 | 15.2 | 20 | 27.4 |
| 74 | 15.8 | 13 | 28.1 |
| 14 | 16.1 | 15 | 28.6 |
| 57 | 17.2 | 11 | 29.2 |
| 20 | 18.2 | 12 | 29.8 |

In another embodiment of the present invention a stable crystalline solid comprising Compound I, designated as Compound I form V is provided.

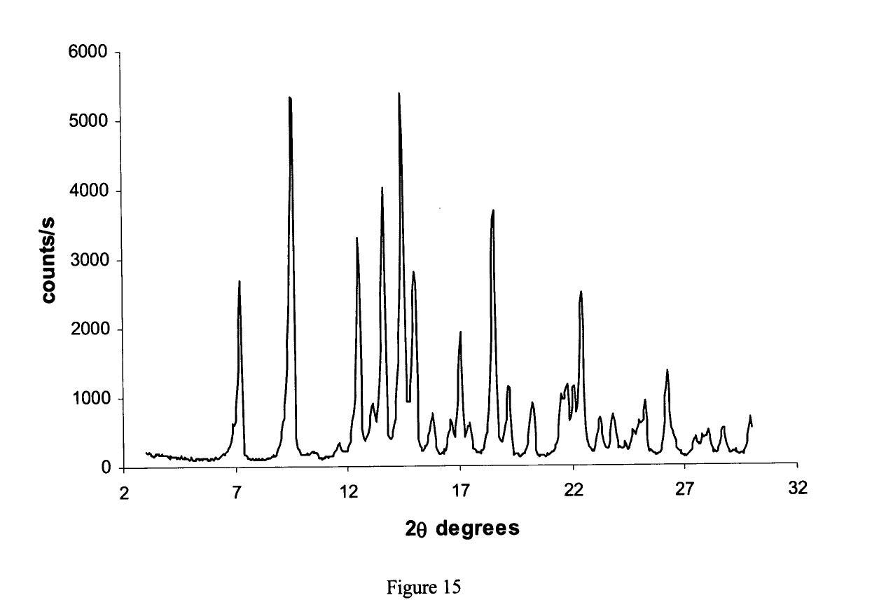

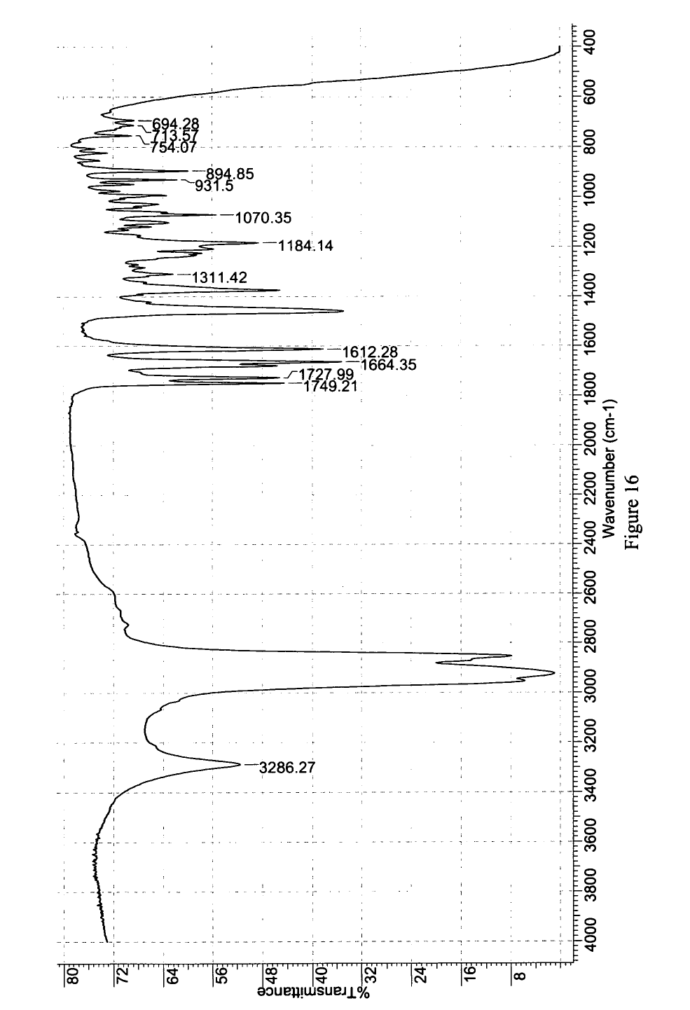

The stable crystalline solid comprising Compound I form V is characterized by unique powder X-ray diffraction pattern (Table 4, FIG. 15). The diffraction peaks at 7.2, 9.6, 12.5, 13.6, 14.5 and 18.5±0.2 degrees 2 θ are most characteristic of this form. It is further characterized by a unique infra-red spectrum (FIG. 16). The peaks at 3286, 1749, 1728 and the singlet at 1612±4 cm⁻¹ are most characteristic of this form.

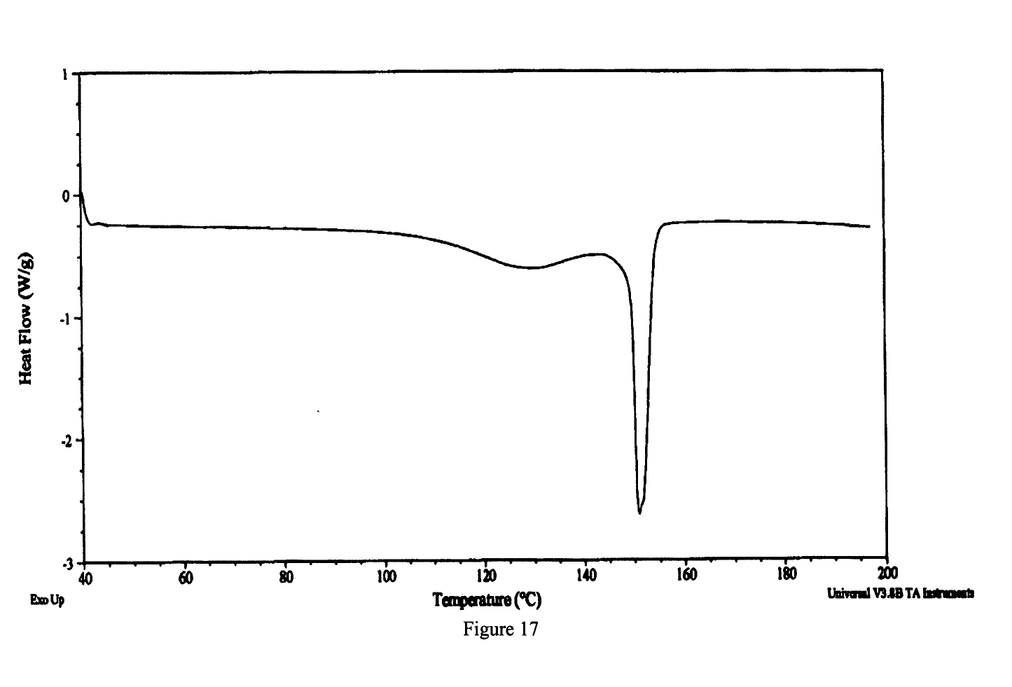

The stable crystalline solid comprising Compound I form V is further characterized by Diffrential Scanning Calorimetry (DSC). DSC analysis of the stable crystalline solid comprising Compound I form V (FIG. 17) shows an endothermic peak at temperatures of between 100° C. to 160° C.

In another embodiment of the present invention, a process for preparing a stable crystalline solid comprising Compound I form V is provided. The process comprising the steps of: (a) dissolving Compound I in acetone to form a solution; (b) crystallizing Compound I Form V from the solution; and (c) isolating the Compound I Form V.

In another embodiment of the present invention, Compound I is added into stirred acetone to form a suspension. The suspension is heated to form a solution. Preferably, the suspension is heated to about reflux temperature, thus making Compound I substantially soluble in acetone.

In yet another embodiment of the present invention, the solution is kept under reflux from about several minutes to about several hours. Preferably the solution is kept under reflux for several minutes.

In another embodiment of the present invention, the solution is cooled to about room temperature.

In yet another embodiment of the present invention, the cooled solution is kept at room temperature until precipitation occurs.

In yet another embodiment of the present invention, Compound I form V is separated by techniques well- known in the art.

In yet another embodiment of the present invention, the isolated Compound I form V can be dried using conventionally known methods. Preferably, the isolated Compound I form V may be dried by placing it in a hood.

In yet another embodiment of the present invention the stable crystalline solid comprising Compound I form V, obtained by the process disclosed hereinabove, is a solvate of acetone.

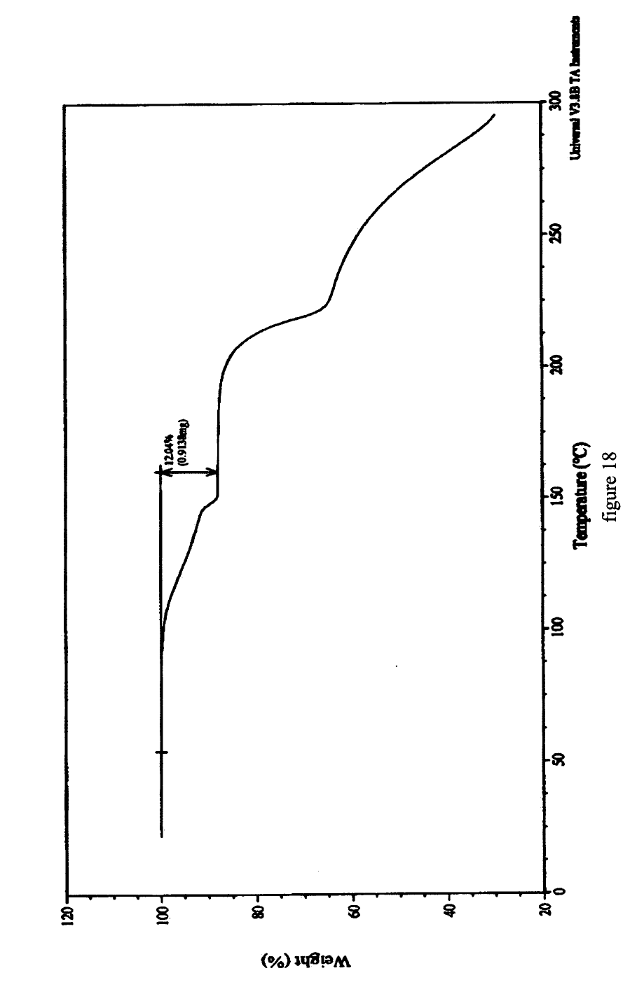

In yet another embodiment of the present invention the stable crystalline solid comprising Compound I form V, obtained by the process disclosed hereinabove, contains acetone in amount of between about 10% to about 12% w/w. Weight loss of from about 10% to about 12% w/w is observed in thermo gravimetric analysis (TGA) (FIG. 18), and the identity of the released solvent was independently determined using head-space GC analysis.

TABLE 4
Form V - Powder X-ray diffraction peak positions and intensities

| Rel. | Peak | Rel. | Peak |
|---|---|---|---|
| Intensity | Position | Intensity | Position |
| (%) | (2θ deg) | (%) | (2θ deg) |
| 50 | 7.2 | 19 | 21.5 |
| 100 | 9.6 | 22 | 21.8 |
| 4 | 10.4 | 21 | 22.0 |
| 7 | 11.5 | 46 | 22.4 |
| 61 | 12.5 | 13 | 23.2 |
| 16 | 13.1 | 13 | 23.8 |
| 78 | 13.6 | 10 | 24.6 |
| 100 | 14.5 | 12 | 25.0 |
| 52 | 15.0 | 17 | 25.2 |
| 14 | 15.8 | 25 | 26.3 |
| 12 | 16.6 | 8 | 27.5 |
| 35 | 17.1 | 8 | 27.7 |
| 12 | 17.4 | 9 | 28.1 |
| 69 | 18.5 | 10 | 28.7 |
| 21 | 19.2 | 13 | 29.9 |
| 17 | 20.2 | 8 | 30.3 |

In another embodiment of the present invention a stable crystalline solid comprising Compound I, designated as Compound I form VI is provided.

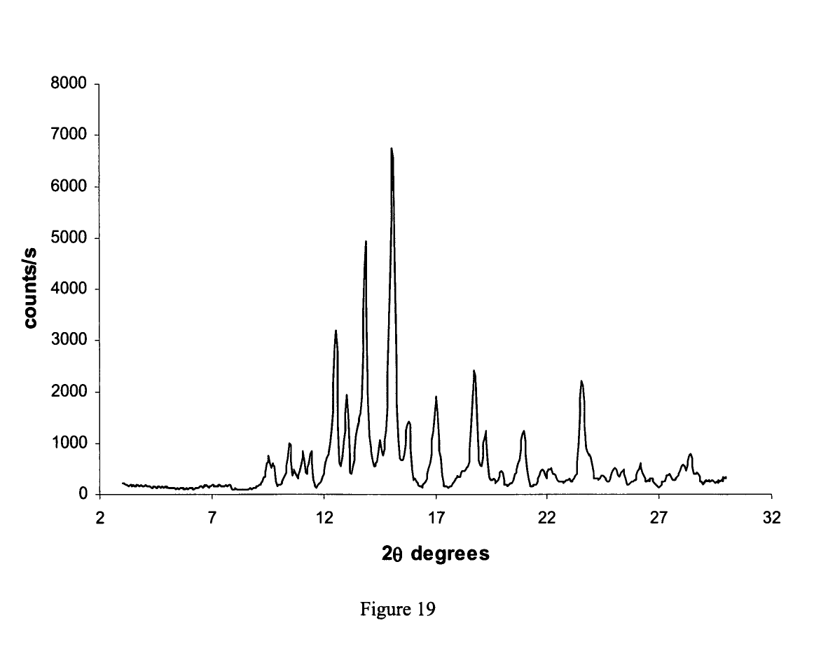

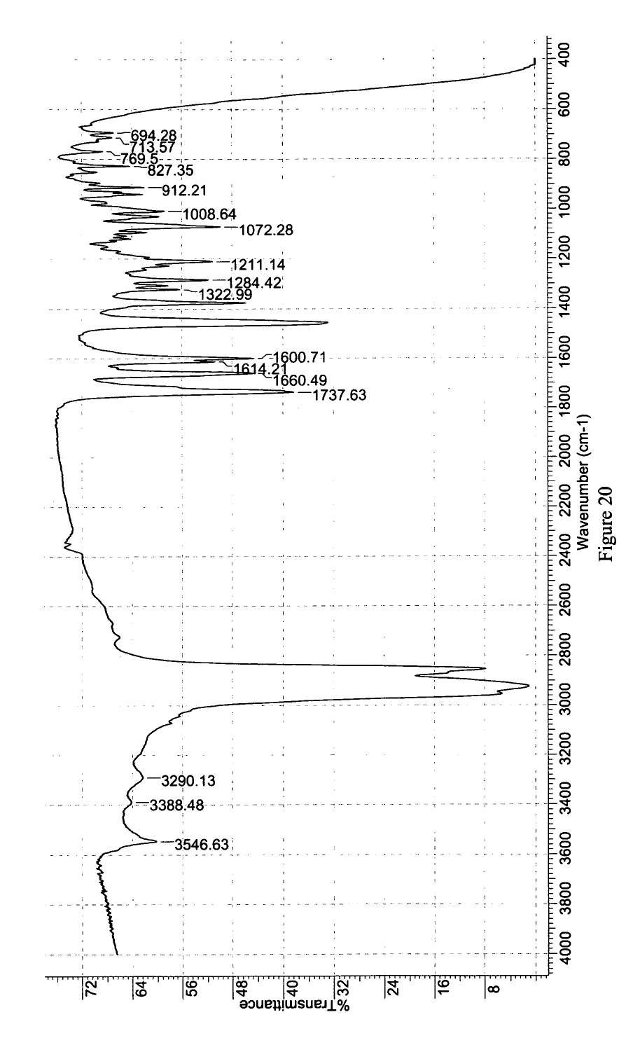

The stable crystalline solid comprising Compound I form VI is characterized by unique powder X-ray diffraction pattern (Table 5, FIG. 19). The diffraction peaks at 6.6, 7.7, 12.6, 13.9, 15.1 and 18.8±0.2 degrees 2 θ are most characteristic of this form. It is characterized by a unique infra-red spectrum (FIG. 20). The absorption peaks at 3547, 3388, 3290 and 1738 (broad) ±4 cm⁻¹ are most characteristic of this form.

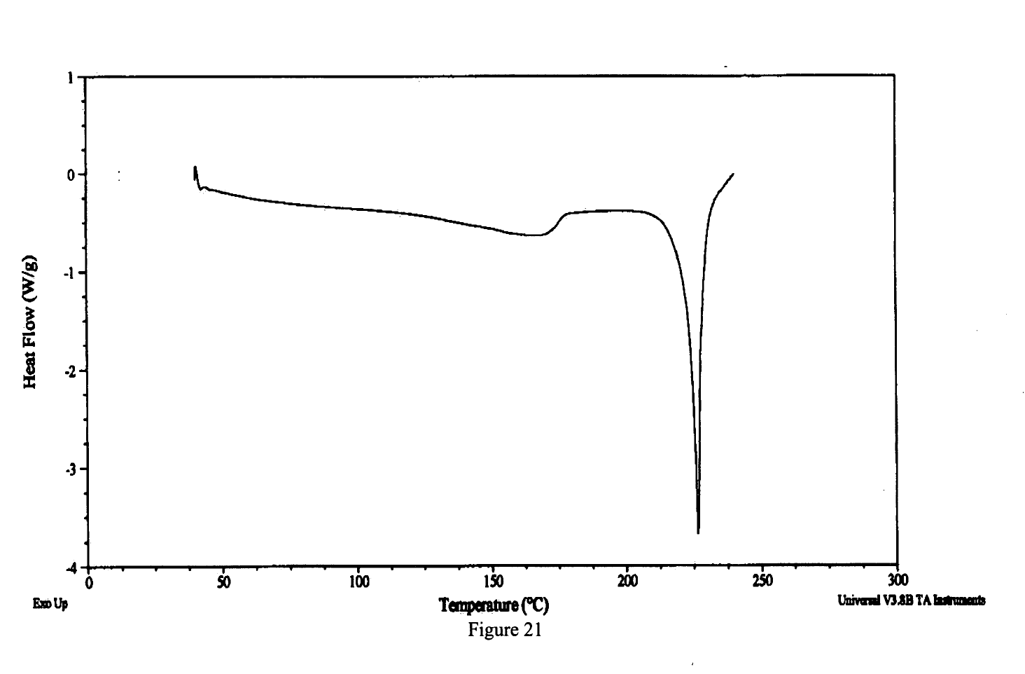

The stable crystalline solid comprising Compound I form VI is further characterized by Differential Scanning Calorimetry (DSC). DSC analysis of Compound I form VI (FIG. 21) shows an endothermic peak at temperatures of between 150° C. to 160° C.

In another embodiment of the present invention, a process for preparing a stable crystalling solid comprising Compound I form VI is provided. The process comprising the steps of: (a) dissolving Compound I in isopropanol to form a solution; (b) crystallizing Compound I Form VI from the solution by rapid removal of the solvent; and (c) isolating the Compound I Form VI.

In another embodiment of the present invention, Compound I is added into stirred isopropanol to form a suspension. The suspension is heated to form a solution. Preferably, the suspension is heated to about reflux temperature, thus making Compound I substantially soluble in isopropanol.

After few minutes under reflux, the solvent is rapidly removed by using one of the known in the art drying technologies or devices including rotary evaporation, vacuum ovens, tray ovens, rotary ovens, and fluidized bed dryers, preferably by using rotary evaporation. The evaporation is conducted under reduced pressure at elevated temperature, preferably at about 40° C., to obtain Compound I form VI.

In another embodiment of the present invention, a second process for preparing a stable crystalline solid comprising Compound I form VI is provided. The process comprising the steps of: (a) dissolving Compound I in a mixture comprising of isopropanol and a non-polar anti-solvent to form a solution; (b) crystallizing Compound I Form VI from the solution; and (c) isolating the Compound I Form VI.

In another embodiment of the present invention, Compound I is added into a stirred mixture of isopropanol and a non-polar anti-solvent to form a suspension. The suspension is heated to form a solution. Preferably, the suspension is heated to about reflux temperature, thus making Compound I substantially soluble in the mixture of isopropanol and the non-polar anti-solvent.

The term “non-polar anti-solvent” is defined as any solvent in which Compound I is poorly soluble in. Preferably, the non-polar anti-solvents, usable in context of the present invention, are hydrocarbons having 5 to 10 carbon atoms, or mixtures thereof, more preferably the non-polar anti-solvent is cyclohexane.

In yet another embodiment of the present invention, the solution is kept under reflux from about several minutes to about several hours. Preferably the solution is kept under reflux for several minutes.

In another embodiment of the present invention, the solution is cooled to about room temperature.

In yet another embodiment of the present invention, the cooled solution is kept at room temperature until precipitation occurs.

In yet another embodiment of the present invention, Compound I form VI is separated by techniques well-known in the art.

In yet another embodiment of the present invention, the isolated Compound I form VI can be dried using conventionally known methods, preferably, the isolated Compound I form VI is dried by placing it in a hood or by evaporating the solvent using a rotary evaporation at 40° C. in vacuum.

In yet another embodiment of the present invention, the stable crystalline solid comprising Compound I form VI, obtained by the processes disclosed hereinabove, is a solvate of isopropanol.

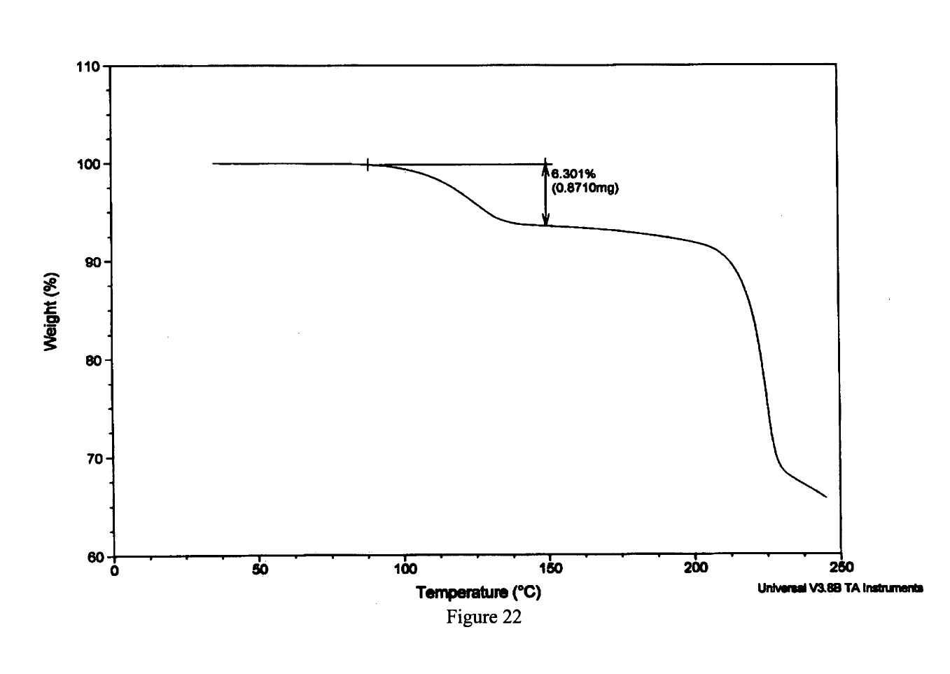

In yet another embodiment of the present invention the stable crystalline solid comprising Compound I form VI, obtained by the process disclosed hereinabove, contains isopropanol in an amount of between about 6% to about 8% w/w. Weight loss of from about 6% to about 8% w/w is observed in thermo gravimetric analysis (TGA) (FIG. 22), and the identity of the released solvent was independently determined using head-space GC analysis.

TABLE 5
Form VI - Powder X-ray diffraction peak positions and intensities

| Rel. | Peak | Rel. | Peak |
|---|---|---|---|
| Intensity | Position | Intensity | Position |
| (%) | (2θ deg) | (%) | (2θ deg) |
| 3 | 6.6 | 18 | 20.9 |
| 3 | 7.7 | 7 | 21.8 |
| 11 | 9.6 | 7 | 22.2 |
| 9 | 9.8 | 32 | 23.6 |
| 15 | 10.5 | 6 | 24.4 |
| 12 | 11.1 | 8 | 25.0 |
| 12 | 11.4 | 7 | 25.4 |
| 46 | 12.6 | 9 | 26.2 |
| 28 | 13.1 | 5 | 26.7 |
| 73 | 13.9 | 6 | 27.5 |
| 16 | 14.5 | 9 | 28.0 |
| 100 | 15.1 | 12 | 28.4 |
| 21 | 15.8 | 7 | 28.7 |
| 28 | 17.0 | 4 | 29.2 |
| 35 | 18.8 | 5 | 29.9 |
| 18 | 19.3 | 5 | 30.3 |
| 7 | 20.0 | | |

In another embodiment of the present invention a stable crystalline solid comprising Compound I, designated as Compound I form VII is provided.

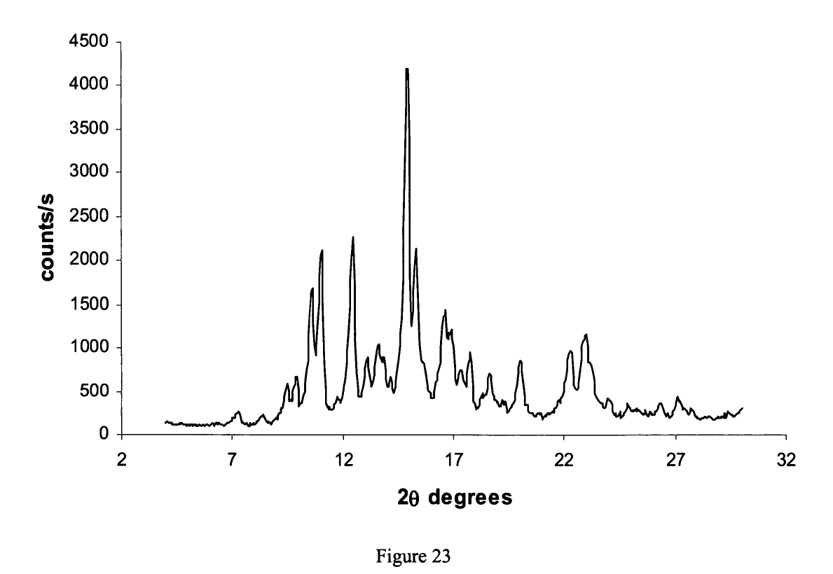

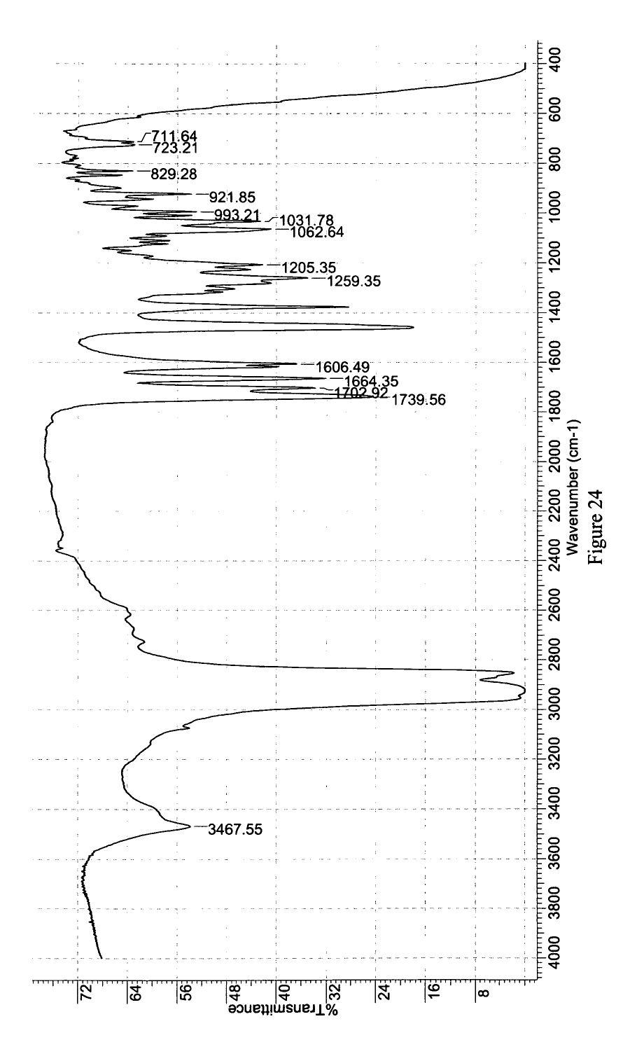

The stable crystalline solid comprising Compound I form VII is characterized by unique powder X-ray diffraction pattern (Table 6, FIG. 23). The diffraction peaks at 10.6, 11.0, 12.4, 14.9, 22.3 and 23.0±0.2 degrees 2 θ are most characteristic of this form. It is further characterized by a unique infra-red spectrum (FIG. 24). The absorption peaks at 3468, 1740, 1703, 1063 and 1032±4 cm⁻¹ are most characteristic of this form.

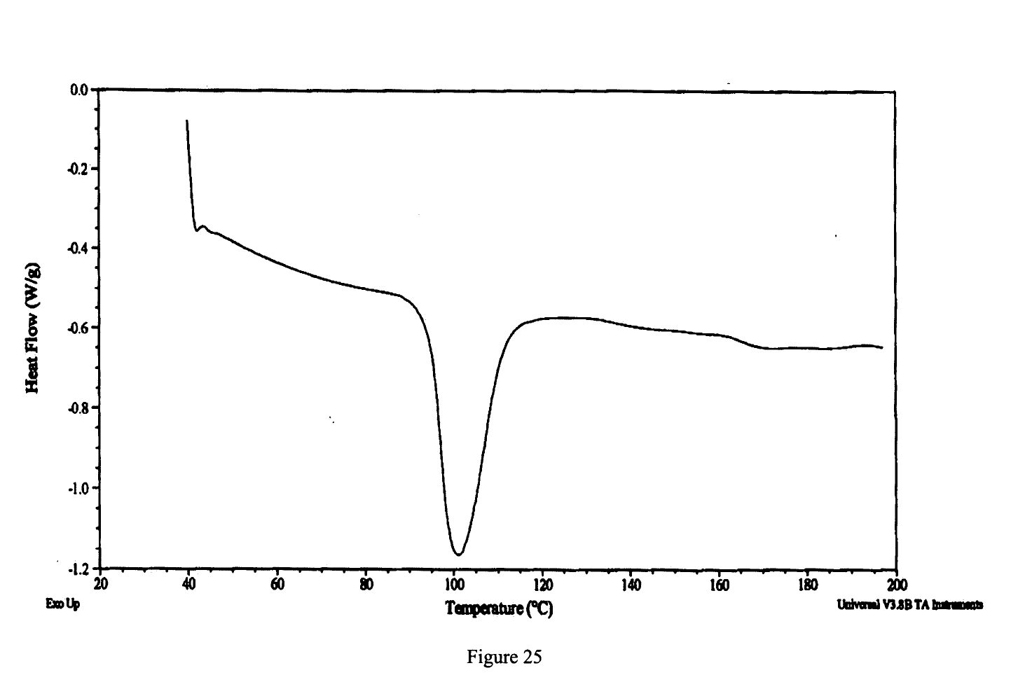

The stable crystalline solid comprising Compound I form VII is further characterized by Differential Scanning Calorimetry (DSC). DSC analysis of form VII (FIG. 25) shows an endothermic peak around 100° C.

In another embodiment of the present invention, a process for preparing a stable crystalline solid comprising Compound I form VI is provided. The process comprising the steps of: (a) dissolving Compound I in ethyl acetate to form a solution; (b) crystallizing Compound I Form VII from the solution by rapid removal of the solvent; and (c) isolating Compound I Form VII.

In another embodiment of the present invention, Compound I is added into stirred ethyl acetate to form a suspension. The suspension is heated to form a solution. Preferably, the suspension is heated to about reflux temperature, thus making Compound I substantially soluble in ethyl acetate.

After few minutes under reflux, the solvent is rapidly removed by evaporation, which is conducted under reduced pressure at elevated temperature, preferably at about 40° C., to obtain Compound I form VII.

In yet another embodiment of the present invention, the stable crystalline solid comprising Compound I form VII, obtained by the process disclosed hereinabove, is a solvate of ethyl acetate.

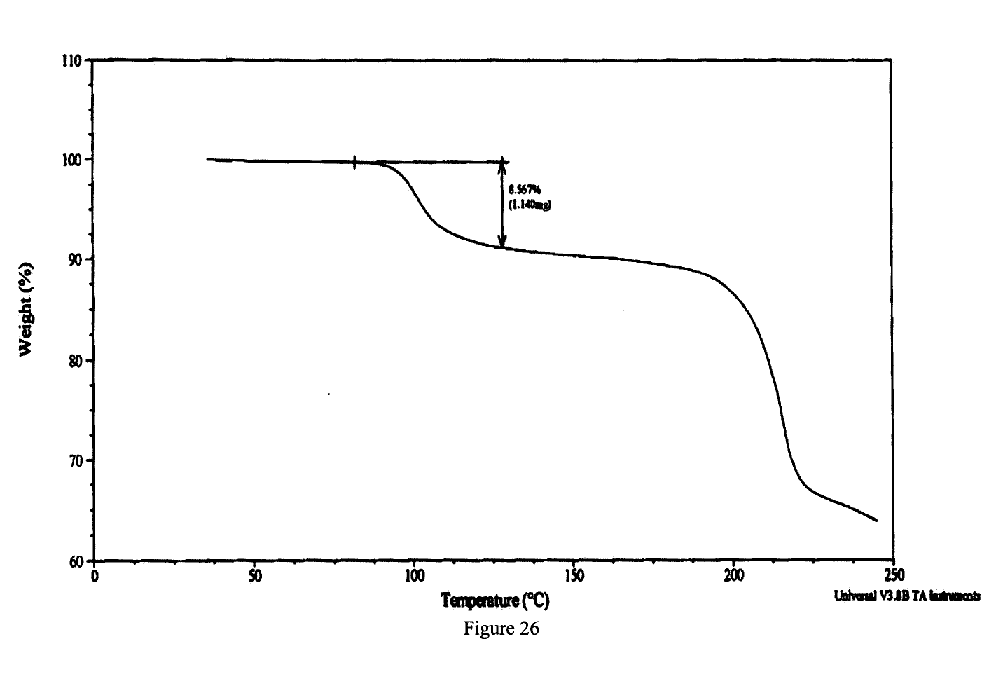

In yet another embodiment of the present invention the stable crystalline solid comprising Compound I form VII, obtained by the process disclosed hereinabove, contains isopropanol in amount of between about 8% to about 14% w/w. Weight loss of from about 8% to about 14% w/w is observed in thermo gravimetric analysis (TGA) (FIG. 26), and the identity of the released solvent was independently determined using head-space GC analysis.

TABLE 6
Form VII - Powder X-ray diffraction peak positions and intensities

| Rel. | Peak | Rel. | Peak |
|---|---|---|---|
| Intensity | Position | Intensity | Position |
| (%) | (2θ deg) | (%) | (2θ deg) |
| 6 | 7.2 | 18 | 17.3 |
| 5 | 8.3 | 22 | 17.7 |
| 14 | 9.5 | 12 | 18.2 |
| 16 | 9.9 | 17 | 18.6 |
| 40 | 10.6 | 10 | 19.2 |
| 51 | 11.0 | 20 | 20.0 |
| 11 | 11.7 | 23 | 22.3 |
| 54 | 12.4 | 27 | 23.0 |
| 21 | 13.1 | 10 | 24.0 |
| 25 | 13.6 | 9 | 24.8 |
| 21 | 13.8 | 7 | 25.2 |
| 16 | 14.1 | 9 | 26.4 |
| 100 | 14.9 | 10 | 27.1 |
| 51 | 15.3 | 7 | 27.7 |
| 34 | 16.6 | 7 | 29.3 |
| 28 | 16.9 | 12 | 30.2 |

In another embodiment of the present invention a stable crystalline solid comprising Compound I, designated as Compound I form VIII is provided.

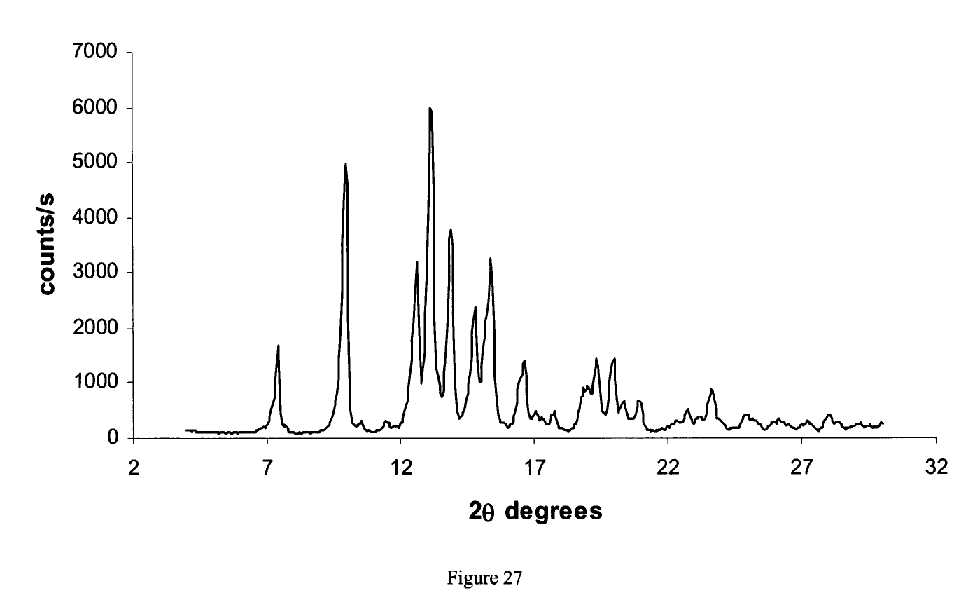

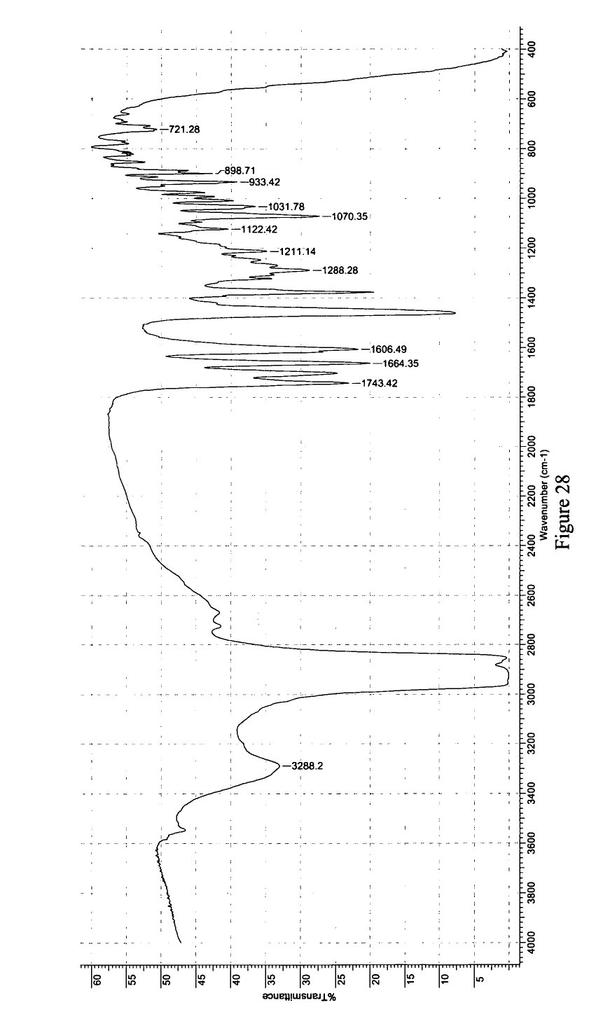

The stable crystalline solid comprising Compound I form VIII is characterized by unique powder X-ray diffraction pattern (Table 7, FIG. 27). The diffraction peaks at 7.4, 10.0, 13.2, 13.9 and 15.4±0.2 degrees 2 θ are most characteristic of this form. It is further characterized by a unique infra-red spectrum (FIG. 28). The absorption peaks at 3288, 1743, 1702, 1664 (singlet) and the doublet around 895±4 cm⁻¹ are most characteristic of this form.

The stable crystalline solid comprising Compound I form VIII is further characterized by having a melting point of 223-225° C.

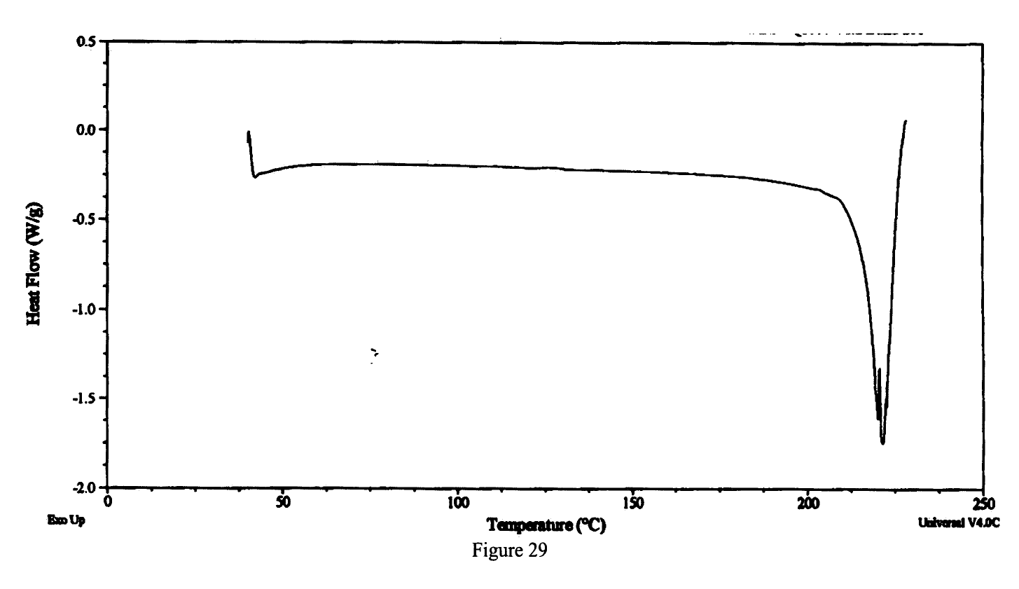

The stable crystalline solid comprising Compound I form VIII is further characterized by Differential Scanning Calorimetry (DSC). DSC curve of Compound I Form VIII (FIG. 29) shows only the melting peak around 223° C. with consequent decomposition.

In another embodiment of the present invention, a process for preparing a stable crystalline solid comprising Compound I form VIII is provided. The process comprising the step of heating Compound I form II, or form V, or form VI, or form VII.

In another embodiment of the present invention the stable crystalline solid comprising Compound I form VIII was prepared by heating Compound I form II, using conventionally known methods, from about several minutes to about several hours, preferably Compound I form II is heated in an oven for several minutes, more preferably Compound I form II is heated in an oven for 15 minutes and most preferably, Compound I form II is heated in an oven at 150° C. for 15 minutes.

In another embodiment of the present invention the stable crystalline solid comprising Compound I form VIII was prepared by heating Compound I form V using conventionally known methods from about several minutes to about several hours, preferably Compound I form V is heated in an oven for several minutes, more preferably Compound I form V is heated in an oven for 15 minutes, more preferably, Compound I form V is heated in an oven at a temperature of between about 110° C. to about 150° C. for 15 minutes and most preferably, Compound I form V is heated in an oven at 150° C. for 15 minutes.

In another embodiment of the present invention the stable crystalline solid comprising Compound I form VIII was prepared by heating Compound I form VI, using conventionally known methods, from about several minutes to about several hours, preferably Compound I form VI is heated in an oven for several minutes, more preferably Compound I form VI is heated in an oven for 15 minutes, more preferably, Compound I form VI is heated in an oven at a temperature of between about 150° C. to about 160° C. for 15 minutes and most preferably, Compound I form VI is heated in an oven at 150° C. for 15 minutes.

In another embodiment of the present invention the stable crystalline solid comprising Compound I form VIII was prepared by heating Compound I form VII, using conventionally known methods from about several minutes to about several hours, preferably Compound I form VII is heated in an oven for several minutes, more preferably Compound I form VII is heated in an oven for 15 minutes, more preferably, Compound I form VII is heated in an oven at a temperature of between about 100° C. to about 150° C. for 15 minutes and most preferably, Compound I form VII is heated in an oven at 150° C. for 15 minutes.

TABLE 7
Form VIII - Powder X-ray diffraction peak positions and intensities

| Rel. | Peak | Rel. | Peak |
|---|---|---|---|
| Intensity | Position | Intensity | Position |
| (%) | (2θ deg) | (%) | (2θ deg) |
| 28 | 7.4 | 25 | 20.0 |
| 82 | 10.0 | 11 | 20.2 |
| 6 | 10.5 | 11 | 20.9 |
| 5 | 11.5 | 6 | 22.3 |
| 53 | 12.6 | 9 | 22.7 |
| 100 | 13.2 | 7 | 23.2 |
| 63 | 13.9 | 15 | 23.7 |
| 40 | 14.8 | 8 | 25.0 |
| 54 | 15.4 | 6 | 26.1 |
| 23 | 16.6 | 5 | 27.1 |
| 9 | 17.0 | 8 | 28.0 |
| 9 | 17.8 | 5 | 28.4 |
| 16 | 19.0 | 5 | 29.1 |
| 24 | 19.4 | 5 | 30.2 |

In another embodiment of the present invention, novel stable crystalline solid comprising Compound I form II, or form III, or form IV, or form V, or form VI, or form VII or form VIII, or a mixture thereof, which are stable during storage, are provided.

In yet another embodiment of the present invention, the novel stable crystalline solid comprising Compound I form II, or form III, or form IV, or form V, or form VI, or form VII, or form VIII, or a mixture thereof, obtained by any of the processes described hereinabove, have improved shelf life without exhibiting a significant change in the active component's physico-chemical characteristics.

In yet another embodiment of the present invention, the novel stable crystalline solid comprising Compound I form II, or form III, or form IV, or form V, or form VI, or form VII, or form VIII, or a mixture thereof, which are obtained by any of the processes described hereinabove, retain the original physico-chemical characteristics over a period of at least one month, more preferably over a period of 6 months and more preferably over periods exceeding one year.

In yet another embodiment of the present invention, the impurities content in the novel stable crystalline solid comprising Compound I form II, or form III, or form IV, or form V, or form VI, or form VII, or form VIII, or a mixture thereof, which are obtained by any of the processes described hereinabove, does not exceed during the storage period 2%, preferably 1%, and more preferably 0.5% in respect to the total weight of the product.

In another embodiment of the present invention, a method of purifying Compound I is provided. The purification can be achieved by conducting each of the processes disclosed hereinabove.

The impure Compound I as used herein refers to a Compound I isolated from any process conventionally known in the art or to be developed in the future.

In another embodiment of the present invention, any of the novel stable crystalline solids comprising Compound I form II, or form III, or form IV, or form V, or form VI, or form VII, or form VIII, or a mixture thereof, which are obtained by any of the processes described hereinabove may be further used according to the general process described hereinabove in the Scheme or by any method conventionally known in the art for the preparation of highly pure fluticasone propionate.

Although, the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims which follow.

### EXAMPLES

The novel crystalline forms of Compound I have been characterized by powder X-ray diffraction, which produces a fingerprint of the particular crystalline form. Measurements of 2 θ values typically are accurate to within ±0.2 degrees.

X-ray diffraction data were acquired using a PHILIPS X-ray diffractometer model PW1050-70. System description: Kα1=1.54178 Å, voltage 40 kV, current 28 mA, diversion slit=1°, receiving slit=0.2 mm, scattering slit=1° with a Graphite monochromator. Experiment parameters: pattern measured between 2 θ=4° and 2 θ=30° with 0.05° increments; count time was 0.5 second per increment

The novel crystalline forms of Compound I have been further characterized by infra-red spectroscopy run on a Nicolet Avator 360.

The novel crystalline forms of Compound I have been further characterized by differential scanning calorimetry (DSC), run on TA instruments model Q1000, with Universal software version 3.88. Samples were analyzed inside crimped 40 μl Aluminum pans. Heating rate for all samples was 10° C./min.

The novel crystalline forms of Compound I have been further characterized by thermogravimetric analysis run on TA instruments model Q500, with universal software version 3.88. Samples were run inside platinum baskets at heating rate of 10° C./min.

The novel crystalline forms of Compound I have been further characterized by Karl-Fischer analysis, using a Mettler Toledo model DL55 Titrator.

Solvent analyses have been carried out using Agilent 6890 Series GC system, equipped with an FID detector and a split mode injector and PAL head space device. Column: DB-624, 30 m, ID=0.53 mm, film thickness 3 μm (J&W CN 125-1334 was used).

The stability tests were carried out using TSP HPLC system, including P4000 Quaternary Gradient Pump, with maximal pressure of 420 bar, with flow accuracy of ±0.5% at 1 m/min with water, and detector UV 1000, with wavelength accuracy of ±1 nm, Autosampler AS 3000 and Communication Hub SN4000.

### Reference Example 1

### Preparation of 6α,9α-difluoro-11β-hydroxy-16α-methyl-3-oxo-17α-propionyloxyandrosta-1,4-diene-17β-carboxylic Acid (Compound I Hydrate) According to the Example of U.S. Pat. No. 4,335,121

A hydrate of Compound I was prepared in accordance with the method described in Preparation VI of U.S. Pat. No. 4,335,121.

A solution of 6α, 9α-difluoro-11β,17α-dihydroxy-16α-methyl-3-oxoandrosta-1,4-diene-17β-carboxylic acid (4 grams) and triethylamine (5 ml) in dichloromethane (120 ml) was cooled to about 0° C. Propionyl chloride (3.75 ml) was added to the solution with stirring. After 1 hour, dichloromethane (50 ml) was added to the solution and the solution was successively washed with 100 ml of 3% sodium hydrogen carbonate, water, 2 N hydrochloric acid, water and saturated brine. The solvent of the thus-washed organic phase was evaporated using a rotary evaporator at 40° C. under a vacuum of 1 mm Hg. The solid residue was dissolved in acetone (100 ml) and diethylamine (5 ml) was added. After 1 hour at 22° C. the solvent was evaporated using a rotary evaporator at 40° C. under a vacuum of 1 mm Hg and the residual gum was dissolved in water (60 ml). Acidification to pH 1 with 2N hydrochloric acid precipitated a solid, which was collected, washed with water, and dried. The solid was heated in a laboratory oven for 48 hours at 60° C. under vacuum of 1 mm Hg.

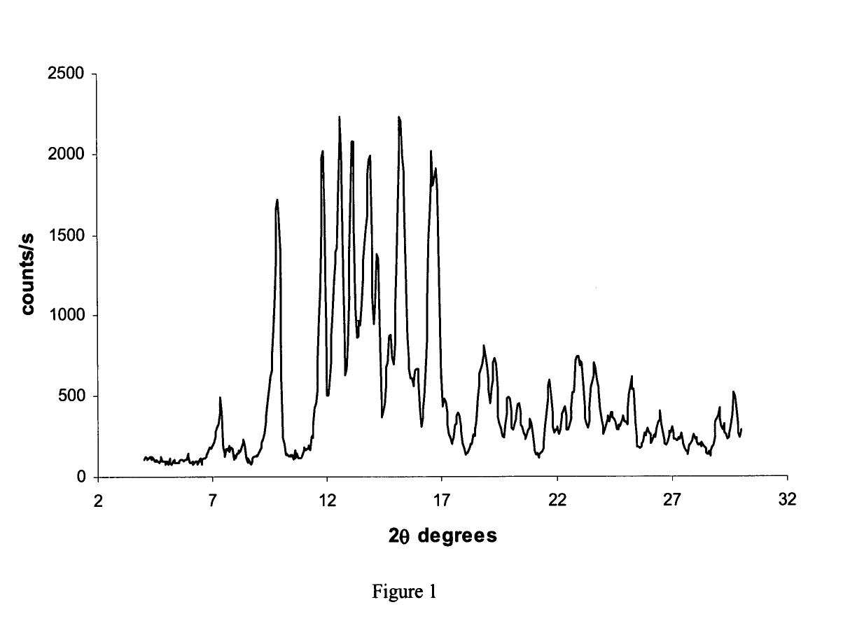

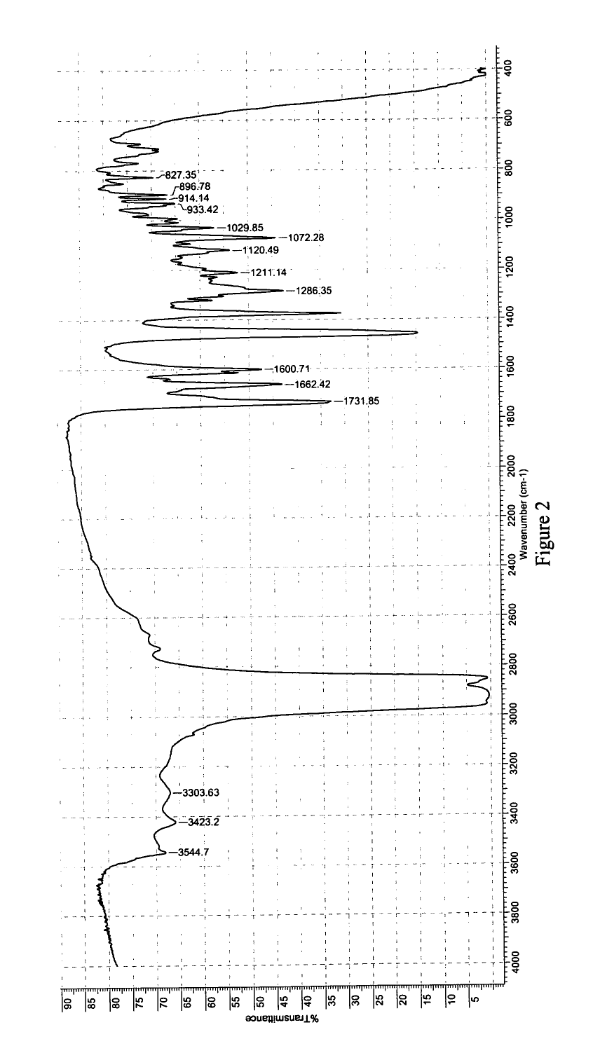

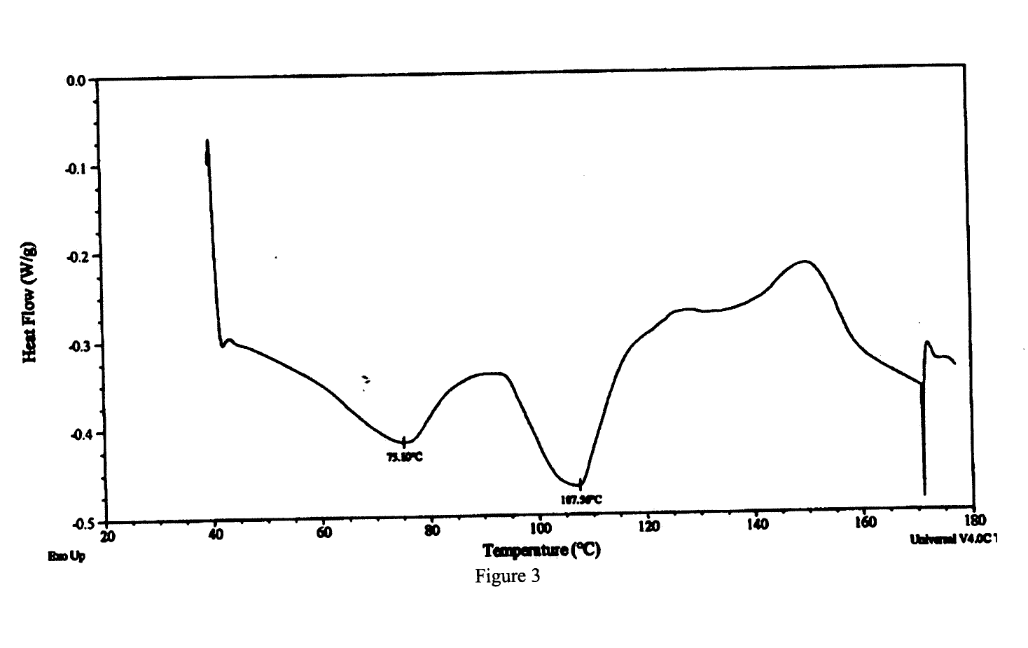

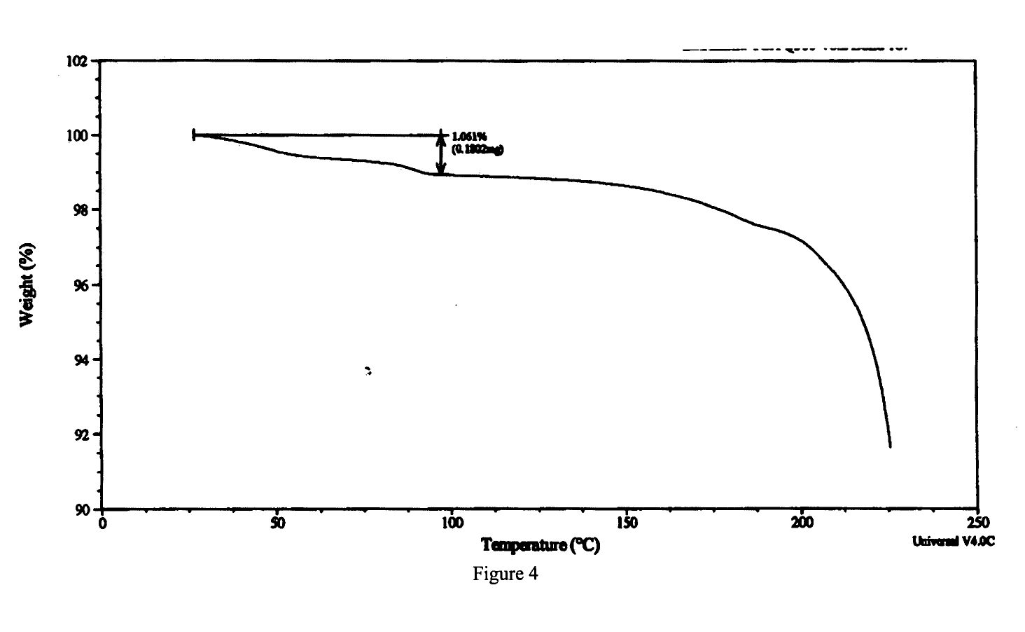

The thus-produced Compound I form I hydrate was analyzed using X-Ray powder diffraction (results depicted in FIG. 1), infrared spectrometry (results depicted in FIG. 2), differential scanning calorimetry (results depicted in FIG. 3) and thermo gravimetric analysis (results depicted in FIG. 4). The water content of the dried product as determined by thermo gravimetric analysis was about 1% by weight.

### Example 1

### Preparation of 6α,9α-difluoro-11β-hydroxy-16α-methyl-3-oxo-17α-propionyloxyandrosta-1,4-diene-17β-carboxylic acid having crystalline form II

In a 250 ml Erlenmeyer flask equipped with a magnetic stirrer, Compound 1 (5 grams) was suspended in 100 ml of acetonitrile. The suspension was left with stirring overnight, and filtered (5.5 grams). The water content was 0.1%. In a stability test that was carried out to a sample of Compound I form II it has been found that the purity of the material (as determined by HPLC) was not changed during a storage period of 1 month, 6 months and even a storage period exceeding one year in a closed vessel at humidity of warehouse conditions and room temperature.

### Example 2

### Preparation of 6α,9α-difluoro-11β-hydroxy-16α-methyl-3-oxo-17α-propionyloxyandrosta-1,4-diene-17β-carboxylic Acid Form III

In a 500 ml three necked round bottom flask equipped with a reflux condenser, a thermometer and a magnetic stirrer, Compound I (1 gram) was suspended in 160 ml of toluene. The suspension was heated to reflux to form a solution, maintained at reflux temperature during few minutes, then left to cool to room temperature. The resulting crystals (0.83 gram) were filtered and left to dry in a hood.

### Example 3

### Preparation of 6α,9α-difluoro-11β-hydroxy-16α-methyl-3-oxo-17α-propionyloxyandrosta-1,4-diene-17β-carboxylic Acid Form III

In a 100 ml three necked round bottom flask equipped with a reflux condenser, a thermometer and a magnetic stirrer, Compound I (1 gram) was suspended in 20 ml of 1-propanol. The suspension was heated to reflux to form a solution, maintained at reflux temperature during few minutes and left to cool to room temperature. The resulting crystals (0.54 gram) were filtered and left to dry in a hood.

### Example 4

### Preparation of 6α,9α-difluoro-11β-hydroxy-16α-methyl-3-oxo-17α-propionyloxyandrosta-1,4-diene-17β-carboxylic Acid Form IV

In a 100 ml three necked round bottom flask equipped with a reflux condenser, a thermometer and a magnetic stirrer, Compound I (2 grams) was suspended in 12 ml of isopropanol. The suspension was heated to reflux to form a solution, maintained at reflux temperature during few minutes and left to cool to room temperature. The resulting crystals (0.75 gram) were filtered and left to dry in a hood.

### Example 5

### Preparation of 6α,9α-difluoro-11β-hydroxy-16α-methyl-3-oxo-17α-propionyloxyandrosta-1,4-diene-17β-carboxylic Acid Form IV

In a 500 ml three necked round bottom flask equipped with a reflux condenser, a thermometer and a magnetic stirrer, Compound I (2 grams) was suspended in 170 ml of ethyl acetate. The suspension was heated to reflux to form a solution, maintained at reflux temperature during few minutes and left to cool to room temperature. The resulting crystals (0.6 gram) were filtered and left to dry in a hood.

### Example 6

### Preparation of 6α,9α-difluoro-11β-hydroxy-16α-methyl-3-oxo-17α-propionyloxyandrosta-1,4-diene-17β-carboxylic Acid Form V

In a 100 ml three necked round bottom flask equipped with a reflux condenser, a thermometer and a magnetic stirrer, Compound I (2 grams) was suspended in 140 ml of acetone. The suspension was heated to reflux to form a solution, maintained at reflux temperature during few minutes and left to cool to room temperature. The resulting crystals (0.7 gram) were collected after few days and left to dry in a hood.

### Example 7

### Preparation of 6α,9α-difluoro-11β-hydroxy-16α-methyl-3-oxo-17α-propionyloxyandrosta-1,4-diene-17β-carboxylic Acid Form VI

In a 100 ml three necked round bottom flask equipped with a reflux condenser, a thermometer and a magnetic stirrer, Compound I (2 grams) was suspended in 12 ml of isopropanol. The suspension was heated to reflux to form a solution, and evaporated using a rotary evaporation at 40° C. in vacuum.

### Example 8

### Preparation of 6α,9α-difluoro-11β-hydroxy-16α-methyl-3-oxo-17α-propionyloxyandrosta-1,4-diene-17βcarboxylic Acid Form VI

In a 100 ml three necked round bottom flask equipped with a reflux condenser, a thermometer and a magnetic stirrer, Compound I (1 gram) was suspended in 60 ml isopropanol:cyclohexane mixture (1:5). The suspension was heated to reflux to form a solution Upon cooling to room temperature, the resulting crystals (0.65 gram) were filtered and dried in air in a hood.

### Example 9

### Preparation of 6α,9α-difluoro-11β-hydroxy-16α-methyl-3-oxo-17α-propionyloxyandrosta-1,4-diene-17β-carboxylic Acid Form VII

In a 500 ml three necked round bottom flask equipped with a reflux condenser, a thermometer and a magnetic stirrer, Compound I (2 grams) was suspended in 170 ml of ethyl acetate. The suspension was heated to reflux to form a solution, and evaporated using a rotary evaporation at 40° C. in vacuum.

### Example 10

### Preparation of 6α,9α-difluoro-11β-hydroxy-16α-methyl-3-oxo-17α-propionyloxyandrosta-1,4-diene-17β-carboxylic Acid Form VIII

1 gram of Compound I form II was heated inside a laboratory oven at 150° C. during 15 minutes.

### Example 11

### Preparation of 6α,9α-difluoro-11β-hydroxy-16α-methyl-3-oxo-17α-propionyloxyandrosta-1,4-diene-17β-carboxylic Acid Form VIII

1 gram of Compound I form V was heated inside a laboratory oven at 150° C. during 15 minutes.

### Example 12

### Preparation of 6α,9α-difluoro-11β-hydroxy-16α-methyl-3-oxo-17α-propionyloxyandrosta-1,4-diene-17βcarboxylic Acid Form VIII

1 gram of Compound I form VI was heated inside a laboratory oven at 150° C. during 15 minutes.

### Example 13

### Preparation of 6α,9α-difluoro-11β-hydroxy-16α-methyl-3-oxo-17α-propionyloxyandrosta-1,4-diene-17β-carboxylic Acid Form VIII

1 gram of Compound I form VII was heated inside a laboratory oven at 150° C. during 15 minutes.

What is claimed is:

1. A stable crystalline solid comprising 6α,9α-difluoro-11β-hydroxy-16α-methyl-3-oxo-17α(-propionyloxy-androsta-1,4-diene-17β-carboxylic acid (Compound I) form II, or form III, or form IV, or form V, or form VI, or form VII, or form VIII, or a mixture thereof containing water in an amount of less than about 2% w/w and preferably, less than about 1% w/w, and most preferably less than about 0.5% w/w, in respect to the total weight of the product.

2. A stable crystalline solid comprising Compound I form II, or form III, or form IV, or form V, or form VI, or form VII, or form VIII, or a mixture thereof, according to claim 1, retaining the original physico-chemical characteristics over a storage period of at least one month, more preferably over a period of at least 6 months and more preferably over a period exceeding one year.

3. A stable crystalline solid comprising Compound I form II, or form III, or form IV, or form V, or form VI, or form VII, or form VIII, or a mixture thereof, according to claim 2, wherein the impurities content during the storage period thereof does not exceed 2%, preferably does not exceed 1%, and more preferably does not exceed 0.5% in respect to the total weight of the product.

4. A stable crystalline solid comprising 6α,9α-difluoro-11β-hydroxy-16α-methyl-3-oxo-17α-propionyloxy-androsta-1,4-diene-17β-carboxylic acid (Compound I) form II, according to claim 3, characterized by unique powder X-ray diffraction pattern, as depicted in table 1 and in FIG. 5, having strong diffraction peaks at 7.3, 9.7, 13.6, 14.6 and 18.6±0.2 degrees 2 θ.

5. A stable crystalline solid comprising Compound I form II, as defined in claim 4, further characterized by having a unique infra-red spectrum, as depicted in FIG. 6, with characterizing absorption peaks at 3315, 3165, 2266, 1732 and 1749±4 cm⁻¹.

6. A stable crystalline solid comprising Compound I form II, as defined in claim 4, further characterized by a differential scanning calorimetric curve having an endothermic peak at about 150° C.

7. A process for preparing a stable crystalline solid comprising Compound I form II, comprising the steps of:
a) dispersing Compound I in acetonitrile; and
b) isolating the Compound I form II.

8. A stable crystalline solid comprising Compound I form II, according to claims 4 and 7, wherein Compound I form II is in the form of a solvate of acetonitrile.

9. A crystalline solid comprising Compound I form II, according to claim 8, wherein the Compound I form II contains acetonitrile in an amount of between about 5% and about 7% w/w, as defined by weight loss of thermo gravimetric analysis (TGA).

10. A stable crystalline solid comprising Compound I form III, according to claim 3, characterized by unique powder X-ray diffraction pattern, as described in table 2 and in FIG. 9, with strong diffraction peaks at 13.9, 15.1, 23.6 and 28.4±0.2 degrees 2 θ.

11. A stable crystalline solid comprising Compound I form III, as defined in claim 10, further characterized by having a unique infra-red spectrum, as depicted in FIG. 10, with characterizing absorption peaks at 1738 and 3547±4 cm⁻¹.

12. A stable crystalline solid comprising Compound I form III, as defined in claim 10, further characterized by a differential scanning calorimetric curve having an endothermic peak at about 230° C. corresponding to its melting and decomposition and a melting point of 232-234° C.

13. A process for preparing a stable crystalline solid comprising Compound I form III, comprising the steps of:
a) dissolving Compound I in a solvent selected from a group consisting of toluene and 1-propanol, to thereby form a solution;
b) crystallizing Compound I Form III from the solution; and
c) isolating the Compound I Form III.

14. A process according to claim 13, wherein Compound I is added into in a solvent selected from a group consisting of toluene and 1-propanol followed by heating the suspension to reflux to thereby form a solution.

15. A stable crystalline solid comprising Compound I form IV, according to claim 3, characterized by having a unique powder X-ray diffraction pattern with strong diffraction peaks at 6.7, 7.6, 7.8, 13.9, 15.8, and 17.2±0.2 degrees 2 θ, as depicted in table 3 and in FIG. 13.

16. A stable crystalline solid comprising Compound I form IV, as defined in claim 15, further characterized by having a unique infra-red spectrum with characterizing absorption peaks at 3477, 3412, 3271, doublet at 1660-1670 and the pattern created by the peaks at 1700-1750±4 cm⁻¹ as depicted in FIG. 14.

17. A stable Compound I form IV, as defined in claim 15, further characterized by a differential scanning calorimetric curve having an endothermic peak at about 220° C. corresponding to its melting and decomposition and by having a melting point of 221-224° C.

18. A process for preparing a stable crystalline solid comprising Compound I form IV, comprising the steps of:
a) dissolving Compound I in a solvent selected from a group consisting of isopropanol and ethyl acetate, to thereby form a solution;
b) crystallizing Compound I Form IV from said solution; and
c) isolating the Compound I Form IV.

19. A process according to claim 18, wherein Compound I is added into in a solvent selected from a group consisting of isopropanol and ethyl acetate followed by heating the suspension to reflux to thereby form a solution

20. A stable crystalline solid comprising Compound I form V, according to claim 3, characterized by unique powder X-ray diffraction pattern with strong diffraction peaks at 7.2, 9.6, 12.5, 13.6, 14.5 and 18.5±0.2 degrees 2 θ, as depicted in table 4 and in FIG. 15.

21. A stable crystalline solid comprising Compound I form V, as defined in claim 20, further characterized by having a unique infra-red spectrum with characterizing absorption peaks at 3286, 1749, 1728 and the singlet at 1612±4 cm⁻¹ as depicted in FIG. 16.

22. A stable crystalline solid comprising Compound I form V, as defined in claim 20, further characterized by a differential scanning calorimetric curve having an endothermic peak at temperatures of between 100° C. to 160° C.

23. A process for preparing stable crystalline solid comprising Compound I form V, comprising the steps of:
a) dissolving Compound I in acetone to thereby form a solution;
b) crystallizing Compound I Form V from said solution; and
c) isolating the Compound I Form V.

24. A process according to claim 23, wherein Compound I is added into the acetone solvent followed by heating the suspension to reflux to thereby form a solution.

25. A stable crystalline solid comprising Compound I form V, according to claims 20 and 24, wherein said Compound I form V is in the form of a solvate of acetone, wherein said solvate contains acetone in an amount of between about 10% and about 12% w/w, as observed by thermo gravimetric analysis (TGA).

26. A stable crystalline solid comprising Compound I form VI, according to claim 3, characterized by having a unique powder X-ray diffraction pattern with strong diffraction peaks at 6.6, 7.7, 12.6, 13.9, 15.1 and 18.8±0.2 degrees 2 θ, as depicted in table 5 and in FIG. 19.

27. A stable crystalline solid comprising Compound I form VI, as defined in claim 26, further characterized by having a unique infra-red spectrum with characterizing absorption peaks at 3547, 3388, 3290 and 1738 (broad) ±4 cm⁻¹, as described in FIG. 20.

28. A stable crystalline solid comprising Compound I form VI, as defined in claim 26, further characterized by a differential scanning calorimetric curve having an endothermic peak at temperatures of between 150° C. to 160° C.

29. A process for preparing a stable crystalline solid comprising Compound I form VI, comprising the steps of:
a) dissolving Compound I in isopropanol to thereby form a solution;
b) crystallizing Compound I Form VI from said solution by rapid removal of said isopropanol; and
c) isolating the Compound I Form VI.

30. A process according to claim 29, wherein Compound I is added into the isopropanol solvent followed by heating the suspension to reflux to thereby form a solution.

31. A process for preparing stable crystalline solid comprising Compound I form VI, comprising the steps of:
a) dissolving Compound I in a mixture comprising isopropanol and a non-polar anti-solvent, preferably cyclohexane, to thereby form a solution;
b) crystallizing Compound I Form VI from said solution; and
c) isolating the Compound I Form VI.

32. A process according to claim 31, wherein said solution of Compound I is obtained by suspending Compound I in said mixture comprising isopropanol and a non-polar anti-solvent followed by heating the suspension to reflux to thereby form a solution.

33. A stable crystalline solid comprising Compound I form VI, according to claims 29 and 31, wherein said Compound I form VI is in the form of a solvate of isopropanol, containing isopropanol in an amount of between about 6% and about 8% w/w, as observed by thermo gravimetric analysis (TGA).

34. A stable crystalline solid comprising Compound I form VII, according to claim 3, characterized by having a unique powder X-ray diffraction pattern with strong diffraction peaks at 10.6, 11.0, 12.4, 14.9, 22.3, and 23.0±0.2 degrees 2 θ, as depicted in table 6 and in FIG. 23.

35. A stable crystalline solid comprising Compound I form VII, as defined in claim 34, further characterized by having a unique infra-red spectrum with characterizing absorption peaks at 3468, 1740, 1703, 1063 and 1032±4 cm⁻¹, as depicted in FIG. 24.

36. A stable crystalline solid comprising Compound I form VII, as defined in claim 34, further characterized by a differential scanning calorimetric curve having an endothermic peak at temperatures around 100° C.

37. A process for preparing a stable crystalline solid comprising Compound I form VII, comprising the steps of:
a) dissolving Compound I in ethyl acetate to thereby form solution;
b) crystallizing Compound I Form VII from said solution by rapid removal of said ethyl acetate; and
c) isolating the Compound I Form VII.

38. A process according to claim 37, wherein said solution of Compound I is obtained by suspending Compound I in ethyl acetate followed by heating the suspension to reflux to thereby form a solution.

39. A stable crystalline solid comprising Compound I form VIII, according to claim 3, characterized by having a unique powder X-ray diffraction pattern with strong diffraction peaks at 7.4, 10.0, 13.2, 13.9 and 15.4±0.2 degrees 2 θ, as depicted in table 7 and in FIG. 27.

40. A stable crystalline solid comprising Compound I form VIII, as defined in claim 39, further characterized by having a unique infra-red spectrum with characterizing absorption peaks at 3288, 1743, 1702, 1664 (singlet) and a doublet around 895±4 cm⁻¹, as described in FIG. 28.

41. A stable crystalline solid comprising Compound I form VIII, as defined in claim 39, further characterized by a differential scanning calorimetric curve having an endothermic peak at temperatures around 223° C. corresponding to its melting and decomposition and a melting point of 223-225° C.

42. A process for preparing a stable crystalline solid comprising Compound I form VIII, comprising the step of heating Compound I form II, or form V, or form VI or form VII.

43. A process according to claim 42, comprising heating stable crystalline solid comprising Compound I form II by using conventionally known methods for a time period that ranges from about several minutes to about several hours, preferably heating Compound I form II in an oven for several minutes, more preferably heating Compound I form II in an oven for 15 minutes and most preferably, heating Compound I form II in an oven for 15 minutes at 150° C.

44. A process according to claim 42, comprising heating a stable crystalline solid comprising Compound I form V by using conventionally known methods for a time period that ranges from about several minutes to about several hours, preferably heating Compound I form V in an oven for several minutes, more preferably heating Compound I form V in an oven for 15 minutes, more preferably, heating Compound I form V in an oven at a temperature of between about 110° C. and about 150° C. for 15 minutes and most preferably, heating Compound I form V in an oven at 150° C. for 15 minutes.

45. A process according to claim 42, comprising heating a stable crystalline solid comprising Compound I form VI by using conventionally known methods for a time period that ranges from about several minutes to about several hours, preferably Compound I form VI is heated by oven for several minutes, more preferably heating Compound I form VI in an oven for 15 minutes, more preferably, heating Compound I form VI in an oven at a temperature of between about 150° C. and about 160° C. for 15 minutes and most preferably, heating Compound I form VI in an oven for 15 minutes at 150° C.

46. A process according to claim 42, comprising heating a stable crystalline solid comprising Compound I form VII by using conventionally known methods for a time period that ranges from about several minutes to about several hours, preferably heating Compound I form VII in an oven for several minutes, more preferably heating Compound I form VII in an oven for 15 minutes, more preferably, heating Compound I form VII in an oven at a temperature of between about 100° C. and about 150° C. for 15 minutes and most preferably, heating Compound I form VII in an oven for 15 minutes at 150° C.

47. Highly pure fluticasone propionate prepared from stable crystalline solid comprising compound I form II, or form III, or form IV, or form V, or form VI, or form VII or form VIII, or a mixture thereof, prepared and isolated essentially as described herein.
